SEQUENCE LISTING (27 sequences)

SEQ ID NO: 1 - protein, 52 aa, Zika virus
IRCIGVSNRDFVEGMSGGTWVDVVLEHGGCVTVMAQDKPTVDIELVTTTVSN

SEQ ID NO: 2 - protein, 58 aa, Zika virus
LEYRIMLSVHGSQHSGMIVNDTGHETDENRAKVEITPNSPRAEATLGGFGSLGLDCEP

SEQ ID NO: 3 - protein, 19 aa, Zika virus
GHLKCRLKMDKLRLKGVSY

SEQ ID NO: 4 - protein, 99 aa, Zika virus
SLCTAAFTFTKIPAETLHGTVTVEVQYAGTDGPCKVPAQMAVDMQTLTPVGRLITANPVITESTENSKMMLELDPPFGDSYIVIGVGEKKITHHWHRSG

SEQ ID NO: 5 - protein, 8 aa, synthetic construct
GGGGGGGG

SEQ ID NO: 6 - protein, 6 aa, synthetic construct
GGGGGG

SEQ ID NO: 7 - protein, 4 aa, synthetic construct
GGGG

SEQ ID NO: 8 - protein, 4 aa, synthetic construct
GGGS

SEQ ID NO: 9 - protein, 5 aa, synthetic construct
GGGGS

SEQ ID NO: 10 - protein, 10 aa, synthetic construct
GGGGSGGGGS

SEQ ID NO: 11 - protein, 15 aa, synthetic construct
GGGGSGGGGSGGGGS

SEQ ID NO: 12 - protein, 20 aa, synthetic construct
GGGGSGGGGSGGGGSGGGGS

SEQ ID NO: 13 - protein, 18 aa, synthetic construct
KESGSVSSEQLAQFRSLD

SEQ ID NO: 14 - protein, 14 aa, synthetic construct
EGKSSGSGSESKST

SEQ ID NO: 15 - protein, 12 aa, synthetic construct
GSAGSAAGSGEF

SEQ ID NO: 16 - protein, 15 aa, synthetic construct
GLNDIFEAQKIEWHE

SEQ ID NO: 17 - protein, 8 aa, synthetic construct
HHHHHHHH

SEQ ID NO: 18 - protein, 8 aa, Enterovirus C
LEVLFQGP

SEQ ID NO: 19 - protein, 249 aa, synthetic construct
IRCIGVSNRDFVEGMSGGTWVDVVLEHGGCVTVMAQDKPTVDIELVTTTVSNGGGGGGGGLEYRIMLSVHGSQHSGMIVNDTGHETDENRAKVEITPNSPRAEATLGGFGSLGLDCEPRTGLGGGGGGGGSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSG

SEQ ID NO: 20 - protein, 277 aa, synthetic construct
IRCIGVSNRDFVEGMSGGTWVDVVLEHGGCVTVMAQDKPTVDIELVTTTVSNGGGGGGGGLEYRIMLSVHGSQHSGMIVNDTGHETDENRAKVEITPNSPRAEATLGGFGSLGLDCEPRTGLGGGGGGGGSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSGGGSGLNDIFEAQKIEWHEHHHHHHHH

SEQ ID NO: 21 - protein, 286 aa, synthetic construct
IRCIGVSNRDFVEGMSGGTWVDVVLEHGGCVTVMAQDKPTVDIELVTTTVSNGGGGGGGGLEYRIMLSVHGSQHSGMIVNDTGHETDENRAKVEITPNSPRAEATLGGFGSLGLDCEPRTGLGGGGGGGGSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGGSLEVLFQGPGGGSGLNDIFEAQKIEWHEHHHHHHHH

SEQ ID NO: 22 - DNA, 834 bp, synthetic construct
  source 1..834: mol_type = other DNA
atccgctgcatcggcgtgtccaaccgcgacttcgtggagggcatgagcggcggcacctgggtggatgtggtgctggagcacggtggctgcgtgaccgtgatggcccaggacaagcccaccgtggatatcgagctggtgaccaccaccgtgtccaacggaggcggcggtggcggaggcggtctggagtaccgcatcatgctgtccgtgcacggctcccagcatagcggcatgatcgtgaacgacaccggccacgagaccgatgagaaccgcgccaaggtggagatcacccccaacagcccccgcgccgaggccaccctgggcggcttcggcagcctgggcctggactgcgagccccgcaccggcctgggcggcggtggaggcggaggtggcagcggccatctgaagtgccgcctgaagatggataagctgcgcctgaagggcgtgtcctacagcctgtgcaccgccgccttcaccttcaccaagattcccgccgagaccctgcatggcaccgtgaccgtggaggtgcagtacgccggcaccgatggcccctgcaaggtgcccgcccagatggccgtggatatgcagaccctgacccccgtgggccgcctgatcaccgccaaccccgtgatcaccgagtccaccgagaacagcaagatgatgctggagctggacccgcccttcggcgattcctacatcgtgatcggcgtgggcgagaagaagatcacccaccattggcatcgctccggctccggcggcggcagcggcctgaacgatatcttcgaggcccagaagatcgagtggcatgagcaccatcatcaccaccatcaccattga

SEQ ID NO: 23 - DNA, 828 bp, synthetic construct
  source 1..828: mol_type = other DNA
atcaggtgcatcggcgtgagcaatcgcgacttcgtggagggcatgtctggcggcacctgggtggatgtggtgctggagcacggcggctgcgtgacagtgatggcccaggacaagccaaccgtggacatcgagctggtgaccacaaccgtgtccaacggcggaggcggaggcggaggcggactggagtacaggatcatgctgagcgtgcacggcagccagcactccggcatgatcgtgaacgacacaggccacgagacagatgagaatagggccaaggtggagatcacacctaactccccaagggcagaggccaccctgggcggattcggctctctgggcctggactgcgagcctaggacaggcctgggcggaggcggaggcggaggcggaagcggccacctgaagtgccggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggccggctgatcaccgcaaatccagtgatcacagagtctaccgagaacagcaagatgatgctggagctggacccccctttcggcgattcctatatcgtgatcggcgtgggcgagaagaagatcacccaccactggcacagatccggaggcggatctggcctgaacgacatctttgaggcccagaagatcgagtggcacgagcaccatcaccatcaccatcaccattga

SEQ ID NO: 24 - DNA, 747 bp, synthetic construct
  source 1..747: mol_type = other DNA
atcaggtgcatcggcgtgagcaatcgcgacttcgtggagggcatgtctggcggcacctgggtggatgtggtgctggagcacggcggctgcgtgacagtgatggcccaggacaagccaaccgtggacatcgagctggtgaccacaaccgtgtccaacggcggaggcggaggcggaggcggactggagtacaggatcatgctgagcgtgcacggcagccagcactccggcatgatcgtgaacgacacaggccacgagacagatgagaatagggccaaggtggagatcacacctaactccccaagggcagaggccaccctgggcggattcggctctctgggcctggactgcgagcctaggacaggcctgggcggaggcggaggcggaggcggaagcggccacctgaagtgccggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggccggctgatcaccgcaaatccagtgatcacagagtctaccgagaacagcaagatgatgctggagctggacccccctttcggcgattcctatatcgtgatcggcgtgggcgagaagaagatcacccaccactggcacagatccgga

SEQ ID NO: 25 - DNA, 834 bp, synthetic construct
  source 1..834: mol_type = other DNA
atcaggtgcatcggcgtgagcaatcgcgacttcgtggagggcatgtctggcggcacctgggtggatgtggtgctggagcacggcggctgcgtgacagtgatggcccaggacaagccaaccgtggacatcgagctggtgaccacaaccgtgtccaacggcggaggcggaggcggaggcggactggagtacaggatcatgctgagcgtgcacggcagccagcactccggcatgatcgtgaacgacacaggccacgagacagatgagaatagggccaaggtggagatcacacctaactccccaagggcagaggccaccctgggcggattcggctctctgggcctggactgcgagcctaggacaggcctgggcggaggcggaggcggaggcggaagcggccacctgaagtgccggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggccggctgatcaccgcaaatccagtgatcacagagtctaccgagaacagcaagatgatgctggagctggacccccctttcggcgattcctatatcgtgatcggcgtgggcgagaagaagatcacccaccactggcacagatccggatccggaggcggatctggcctgaacgacatctttgaggcccagaagatcgagtggcacgagcaccatcaccatcaccatcaccattga

SEQ ID NO: 26 - DNA, 861 bp, synthetic construct
  source 1..861: mol_type = other DNA
atcaggtgcatcggcgtgagcaatcgcgacttcgtggagggcatgtctggcggcacctgggtggatgtggtgctggagcacggcggctgcgtgacagtgatggcccaggacaagccaaccgtggacatcgagctggtgaccacaaccgtgtccaacggcggaggcggaggcggaggcggactggagtacaggatcatgctgagcgtgcacggcagccagcactccggcatgatcgtgaacgacacaggccacgagacagatgagaatagggccaaggtggagatcacacctaactccccaagggcagaggccaccctgggcggattcggctctctgggcctggactgcgagcctaggacaggcctgggcggaggcggaggcggaggcggaagcggccacctgaagtgccggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggccggctgatcaccgcaaatccagtgatcacagagtctaccgagaacagcaagatgatgctggagctggacccccctttcggcgattcctatatcgtgatcggcgtgggcgagaagaagatcacccaccactggcacagatccggaggatccctggaggtgctgttccagggcccaggaggcggatctggcctgaacgacatctttgaggcccagaagatcgagtggcacgagcaccatcaccatcaccatcaccattga

SEQ ID NO: 27 - protein, 505 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSAD

(19) United States
(12) Patent Application Publication
(10) Pub. No.: US 2025/0381262 A1
(43) Pub. Date: Dec. 18, 2025

(54) FLAVIVIRUS IMMUNOGENS AND VACCINE COMPOSITIONS AND METHODS OF USING THE SAME

(71) Applicant: THE HENRY M. JACKSON FOUNDATION FOR THE ADVANCEMENT OF MILITARY MEDICINE, INC., Bethesda, MD (US)

(72) Inventors: Vincent DUSSUPT, Clarksville, MD (US); Shelly KREBS, Clarksville, MD (US)

(21) Appl. No.: 18/866,371

(22) PCT Filed: May 17, 2023
(86) PCT No.: PCT/US2023/067128

§ 371 Date: Nov. 15, 2024

Related U.S. Application Data
(60) Provisional application No. 63/342,720, filed on May 17, 2022.

(51) Int. Cl. A61K 39/12; A61K 39/00; A61P 31/14; C07K 14/005; C12N 7/00; G01N 33/569; G01N 33/68

(52) U.S. Cl. CPC A61K 39/12; A61P 31/14; C07K 14/005; C12N 7/00; G01N 33/56966; G01N 33/56983; G01N 33/6857; A61K 2039/543; A61K 2039/544; C07K 2319/00; C12N 2770/24122; C12N 2770/24134; G01N 2333/183

(57) ABSTRACT

This application relates generally to flavivirus immunogens and to methods and compositions related thereto. More particularly, the disclosure relates to compositions and methods for the preparation, production, and administration of flavivirus immunogens comprising modified E proteins, including, for example, compositions for use as vaccines against flavivirus and for capturing antibodies against flavivirus.

### CROSS-REFERENCE TO RELATED APPLICATIONS

This application claims priority to U.S. Provisional Application No. 63/342,720 filed 17 May 2022, the entire contents of which are hereby incorporated by reference in their entirety.

### STATEMENT REGARDING FEDERALLY SPONSORED RESEARCH

This invention was made with government support under W81XWH-07-2-0067 and W81XWH-18-2-0040 awarded by United States Army Medical Research and Development Command, 0130602D16 awarded by the United States Defense Health Agency, and AI155983 awarded by the National Institutes of Health. The government has certain rights in the invention.

### SEQUENCE LISTING

The Sequence Listing associated with this application is filed in electronic format as a XML file and hereby incorporated by reference into the specification in its entirety. The name of the XML file containing the Sequence Listing is HMJ_182_PCT_SL.xml and the size of the text file is 30 KB.

### FIELD

This application relates generally to flavivirus immunogens and to methods and compositions related thereto. More particularly, the disclosure relates to compositions and methods for the preparation, production, and administration of flavivirus immunogens comprising modified E proteins, including, for example, compositions for use as vaccines against flavivirus and for capturing antibodies against flavivirus.

### BACKGROUND

Flavivirus is a genus of positive-strand RNA viruses in the family Flaviviridae. The genus includes the West Nile virus (WNV), dengue virus (DENV), tick-borne encephalitis virus, yellow fever virus (YFV), Zika virus (ZIKV), and several other viruses which may cause encephalitis, as well as insect-specific flaviviruses (ISFs) such as cell fusing agent virus (CFAV), Palm Creek virus (PCV), and Parramatta River virus (PaRV). Six flaviviruses, including ZIKV, tick-borne encephalitis (TBEV), DENV, WNV, Japanese encephalitis (JEV), and YFV, are primarily responsible for some of the most potentially fatal diseases that affect billions of humans in endemic regions.

Flaviviruses, such as ZIKV and DENV, are transmitted mostly by mosquitoes and continue to be a world-wide infectious disease threat. ZIKV, for instance, was responsible for an unprecedented outbreak in Central and South Americas in 2015-2016 and is poised to re-emerge in parts of the world where its mosquito vectors are present. Pregnant women are particularly vulnerable to ZIKV infection as vertical transmission to fetuses was shown to cause miscarriages and abnormalities including microcephaly. There is currently no FDA approved vaccine against ZIKV.

Flavivirus vaccines have been historically based on platforms presenting the full envelope (E) protein (within an inactivated or attenuated whole virus), the main target of neutralizing antibody responses. Vaccine studies using these strategies against DENV have elicited poorly neutralizing antibody responses that cross-react to multiple dengue strains and ZIKV, increasing the potential for antibody-dependent enhancement (ADE) of infection. ADE is a life-threatening phenomenon that is believed to have contributed to enhanced dengue disease observed in pediatric cohorts during the Dengvaxia® (CYD-TDV, Sanofi) clinical trials, resulting in halting of the vaccination program in children (Halstead, S. B., Hum. Vaccin. Immunother., 2018, 14 (9): 2158-2162). Every year approximately 100 million people get infected with DENV, with 40,000 dying of severe disease. Dengvaxia® has only been approved for vaccination against DENV in at-risk 9-16 years old children with laboratory-confirmed evidence of previous dengue infection. There is an increased risk of severe disease associated with vaccination in individuals without prior dengue exposure, likely due to ADE to the fusion loop epitope (FLE). Currently, there is no approved DENV vaccine for children under 9 years old or people over 16.

Accordingly, there is an urgent need of vaccines or vaccine components, such as immunogens, that can induce immunogenicity with production of antibodies having potent cross-neutralizing activity across multiple flaviviruses, including ZIKV and DENV, without ADE responses.

### SUMMARY

Disclosed herein are novel immunogens that can induce immunogenicity with production of antibodies having potent cross-neutralizing activity across multiple flaviviruses, including ZIKV (Zika) and DENV (dengue), as well as compositions, such as vaccines, comprising the same and methods of preparing and using the same. The present disclosure encompasses, in some aspects, the observation that, by removing Domain II of the flavivirus envelope (E) protein, which is highly conserved among flaviviruses and one of the major targets of cross-reactive responses that lead to ADE, the resulting E protein subunit composed of only Domains I and III can fold into a soluble and well-expressed recombinant protein displaying key neutralizing epitopes for flavivirus (e.g., ZIKV and DENV) neutralizing antibodies.

Accordingly, in one aspect, provided herein is an immunogen comprising Formula (I):

$${DI}_{1} - {L}_{1} - {DI}_{2} - {L}_{2} - {DI}_{3} - DIII \qquad ( I )$$

wherein DI₁, DI₂, and DI₃ together form Domain I (DI) of a flavivirus envelope (E) protein, DIII is Domain III (DIII) of the flavivirus E protein, and L₁ and L₂ are each independently a flexible linker, and wherein the immunogen does not comprise Domain II of the flavivirus E protein. In some embodiments, the flavivirus E protein is an E protein of Zika virus (ZIKV), dengue virus (DENV), Japanese Encephalitis Virus (JEV), West Nile Virus (WNV), Yellow Fever Virus (YFV), St Louis Encephalitis Virus (SLEV), Spondweni Virus (SPOV), Usutu Virus (USUV), Ilheus Virus (ILHV), Rocio Virus (ROCV), Wesselsbron Virus (WSLV), Tick Borne Encephalitis Virus (TBEV), Powassan Virus (POWV), Langat Virus (LGTV), Kyasanur Forest Disease (KSD), Alkhurma fever disease (AFD), or Omsk hemorrhagic fever virus (OHFV). In some embodiments, the flavivirus E protein comprises an amino acid sequence at least about 80% identical to the amino acid sequence of SEQ ID NO: 27.

In some embodiments, DI₁ comprises an amino acid sequence at least about 90% identical to SEQ ID NO: 1, DI₂ comprises an amino acid sequence at least about 90% identical to SEQ ID NO: 2, DI₃ comprises an amino acid sequence at least about 90% identical to SEQ ID NO: 3, and DIII comprises an amino acid sequence at least about 90% identical to SEQ ID NO: 4. In some embodiments, DI comprises the amino acid sequence of SEQ ID NO: 1, DI₂ comprises the amino acid sequence of SEQ ID NO: 2, DI₃ comprises the amino acid sequence of SEQ ID NO: 3, and DIII comprises the amino acid sequence of SEQ ID NO: 4.

In some embodiments, L₁ and L₂ each independently comprise one or more glycine residues and have a length of from about 4 to about 20 amino acids. In some embodiments, L₁ and L₂ are each independently selected from SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15. In some embodiments, L₁ and L₂ each comprises the amino acid sequence of SEQ ID NO: 5.

In some embodiments, the immunogens of the disclosure further comprise one or more heterologous peptides linked to the C-terminus of the immunogen. In some embodiments, the one or more heterologous peptides comprise the amino acid sequence of SEQ ID NO: 16, SEQ ID NO: 17, and/or SEQ ID NO: 18.

In some embodiments, the immunogens of the disclosure comprise an amino acid sequence at least about 95% identical to the amino acid sequence of SEQ ID NO: 19, SEQ ID NO: 20, or SEQ ID NO: 21. In some embodiments, the immunogens of the disclosure comprise the amino acid sequence of SEQ ID NO: 19, SEQ ID NO: 20, or SEQ ID NO: 21.

In another aspect, provided herein is a nucleic acid molecule encoding any of the immunogens disclosed herein. In some embodiments, the nucleic acid molecule is a DNA molecule or an RNA molecule. In some embodiments, the RNA molecule is a messenger RNA (mRNA) molecule.

In a further aspect, provided herein is a composition comprising any of the immunogens or any of the nucleic acid molecules disclosed herein. In some embodiments, the composition is an immunogenic composition.

In another aspect, provided herein is a vaccine comprising any of the immunogenic compositions of the disclosure, and a pharmaceutically acceptable carrier. In some embodiments, the vaccine further comprises an adjuvant.

In yet another aspect, provided herein is a method of immunizing a subject against a flavivirus infection, the method comprising administering to the subject in need thereof any of the vaccines disclosed herein. Also disclosed is a method of reducing one or more symptoms of a flavivirus infection, or a method of inducing an immune response in a subject against flavivirus, the method comprising administering to a subject in need thereof any of the vaccines disclosed herein. In some embodiments, the method prevents a flavivirus infection in the subject, decreases the subject's likelihood of getting a flavivirus infection, or reduces the subject's likelihood of getting serious illness from a flavivirus infection. In some embodiments, the method raises a protective immune response in the subject. In some embodiments, the subject is a human. In some embodiments, the vaccine is administered intramuscularly, intradermally, subcutaneously, intravenously, intranasally, by inhalation, or intraperitoneally.

In another further aspect, provided herein is a method of identifying an antibody against flavivirus in a sample, the method comprising: a) contacting a sample with at least one polypeptide comprising an amino acid sequence at least about 95% identical to the amino acid sequence of SEQ ID NO: 19, SEQ ID NO: 20, or SEQ ID NO: 21; and b) determining formation of a complex between the at least one polypeptide and at least one substance in the sample, wherein formation of the complex indicates that the at least one substance is an antibody against flavivirus. Also provided is a method of identifying a B cell lymphocyte expressing an antibody that binds to an antigen of a flavivirus, the method comprising: a) contacting a B cell lymphocyte in a sample with at least one polypeptide comprising an amino acid sequence at least about 95% identical to the amino acid sequence of SEQ ID NO: 19, SEQ ID NO: 20, or SEQ ID NO: 21; and b) determining formation of a complex between the B cell lymphocyte and the at least one polypeptide, wherein formation of the complex indicates that the B cell lymphocyte expresses an antibody that binds to an envelope (E) protein of the flavivirus. In some embodiments, the at least one polypeptide is labeled with one or more chemicals that are able to emit fluorescence and the complex is determined using a fluorescence-activated cell sorting device. In some embodiments, the at least one polypeptide is labeled with one or more chemicals that are able to emit chemiluminescent light, and the complex is determined using a device that is capable of detecting chemiluminescent light. In some embodiments, the sample used in any of these methods is a tissue sample or a body fluid sample, such as blood, plasma, serum, saliva, tear, urine, cerebrospinal fluid, pleural effusion, ascites, or peritoneal effusion.

### BRIEF DESCRIPTION OF THE DRAWINGS

The accompanying drawings, which are incorporated in and constitute a part of this specification, illustrate certain embodiments, and together with the written description, serve to explain certain principles of the compositions and methods disclosed herein.

FIG. 1A-1C depict a representative design of the flavivirus immunogens according to the disclosure. FIG. 1A: Ribbon representation of the ZIKV envelope (E) protein dimer (PDB 5LBV) showing the two Domain II (DII; shade with diagonal line) insertions (1 and 2) into Domain I (DI; white shade). FIG. 1B: The protein structure of the full-length ZIKV E protein (top) and ZIKV DI-DIII immunogen with two glycine linkers (box with one diagonal line) replacing the original DII domain (bottom). FIG. 1C: The protein structure and amino acid sequence of the expressed ZIKV DI-DIII immunogen (SEQ ID NO: 20).

FIG. 2A-2C depict the results of expression and purification of the ZIKV DI-DIII immunogen expressed in Drosophila S2 cells. FIG. 2A: SDS-PAGE of the ZIKV DI-DIII immunogen eluted from Ni-NTA affinity resin. FIG. 2B: SDS-PAGE of the ZIKV DI-DIII immunogen after size exclusion chromatography. FIG. 2C: Comparison of purity and yield across different ZIKV E constructs.

FIG. 3 depicts the binding of the ZIKV DI-DIII immunogen to MZ4-like and DIII-directed mAbs. Binding of several flavivirus neutralizing antibodies was assessed by biolayer interferometry by loading biotinylated ZIKV DI-DIII immunogen on streptavidin sensors. MZ4-like mAbs (MZ4, MZ2) and DIII-directed mAbs (ZKA190 (DIII), Z004 (DIII)) bound strongly to the ZIKV DI-DIII immunogen. No binding was observed with Domain II Fusion Loop Epitope (FLE)-(2A10G6) and EDE-(EDE1-C8) directed antibodies (2A10G6 (FLE)/EDE1).

FIG. 4 depicts high frequency of DI-DIII-specific B cells identified from Zika purified whole-virus (ZPIV)-vaccinated donors. When used as a probe, B cells from ZPIV-vaccinated individuals bound to the ZIKV DI-DIII immunogen with high frequency. ZPIV-vaccinated individuals (donors 1 and 2) and a negative control were stained with ZIKV E full-length and DI-DIII probes. A similar frequency of antigen-specific B cells was identified when using either the ZIKV DI-DIII immunogen or ZIKV full length E as probes.

FIG. 5 depicts a schematic of the immunization schedule used in the in vivo study with IFNAR^{-/-} mice as described in Example 2. Top (Schematic 1): IFNAR^{-/-} mice received the ZIKV full-length E protein (ZIKV E), DI-DIII, or DIII at day 1 (prime) and 21 (boost), and then challenged with a lethal dose of ZIKV at day 55. Bottom (Schematic 2): IFNAR^{-/-} mice received saline (PBS) or the adjuvant (Alum) alone as controls or the ZIKV full-length E protein (ZIKV E), DI-DIII, or DIII at day 1 (prime) and 21 (boost), but no challenge was given at any later time point for immunogenicity study only. Mice received saline or adjuvant alone were used as controls (not shown).

FIG. 6A-6B show that immunization with DI-DIII protects IFNAR^{-/-} mice from lethal ZIKV infections. FIG. 6A: Probability of survival post-challenge across the different groups (n=10 to 15 mice per group). FIG. 6B: Averaged percent weight loss post-challenge across the different groups. Only mice immunized with full-length ZIKV E protein (“sENV”) and DI-DIII (“DI_DIII”) were protected from lethal ZIKV infection and weight loss. “Alum”: mice immunized with the adjuvant (Alum) alone; “PBS”: mice immunized with saline alone; “DIII”: mice immunized with DIII.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure, and should not be interpreted as limiting the scope of the disclosure.

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

### Definitions

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or =0.1% from the specified value as such variations are appropriate to perform the disclosed methods and/or to make and use the disclosed compositions. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “antibody-dependent enhancement” or “ADE,” as used herein, refers to phenomena characterized by non-neutralizing (or sub-optimally neutralizing) antibodies that facilitate virus entry into host cells, leading to increased infectivity in the cells. In some embodiments, ADE refers to a significant, detectable increase in viral infection in the presence of an antibody, relative to a pre-immune sample or an unrelated antibody.

The term “at least,” “less than,” “more than,” or “up to” prior to a number or series of numbers (e.g., “at least two”) is understood to include the number adjacent to the term “at least,” “less than” or “more than,” and all subsequent numbers or integers that could logically be included, as clear from context. When the term “at least,” “less than,” “more than,” or “up to” is present before a series of numbers or a range, it is understood that “at least,” “less than,” “more than,” or “up to” can modify each of the numbers in the series or range.

The term “carrier,” as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are, or include, one or more solid components.

The term “dengue virus” refers to a group of four genetically and antigenically related viruses, namely DENV-1, DENV-2, DENV-3, and DENV-4.

The term “flexible linker,” as used herein, refers to an empirical linker that is usually used to link protein domains which require a certain degree of movement or interaction. Flexible linkers are generally rich in small or polar amino acids such as Gly and Ser, but can contain additional amino acids such as Thr and Ala to maintain flexibility, as well as polar amino acids such as Lys and Glu to improve solubility. The small size of these amino acids provides flexibility and allows for mobility of the connecting functional domains. Not wishing to be bound by any theory, the incorporation of Ser or Thr can maintain the stability of the linker in aqueous solutions by forming hydrogen bonds with the water molecules, and therefore reduce the unfavorable interaction between the linker and the protein moieties.

The term “immunogen,” as used herein, refers to any substance which is capable, under appropriate conditions, of stimulating an immune response, such as the production of antibodies or a T-cell response in an animal, including compositions that are injected or absorbed into an animal.

As used herein, the term “in some embodiments,” “in certain embodiments,” “in other embodiments,” “in some other embodiments,” or the like, refers to embodiments of all aspects of the disclosure, unless the context clearly indicates otherwise.

The term “prevent,” “preventing,” or “prevention,” as used herein, refers to prophylaxis, avoidance of disease manifestation, a delay of onset, and/or reduction in frequency and/or severity of one or more symptoms of a particular disease, disorder or condition (e.g., infection with, for example, a flavivirus, such as ZIKV or DENV). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

As used herein, the term “prophylactically effective amount” means an amount sufficient to avoid disease manifestation, delay onset of and/or reduce in frequency and/or severity one or more symptoms of a particular disease, disorder or condition (e.g., infection with, for example, a flavivirus, such as ZIKV or DENV).

The term “sequence identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Sequence identity” and “sequence similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). Typical methods to determine identity are designed to give the largest match between the sequences tested. Methods to determine sequence identity and similarity are codified in publicly available computer programs. Typical computer program methods to determine identity and similarity between two sequences include, but are not limited to, the GCG program package (Devereux, J., et al., Nucleic Acids Research 12 (1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity. IgBlast may also be used to determine germline V, D and J gene matches to a query sequence, which is available on the world wide web at ncbi.nlm.nih.gov/igblast/. In some embodiments, the sequence identity is determined using the BLAST X program with the default parameters.

As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, the term “individual” or “patient” is used and is intended to be interchangeable with the term “subject.”

### Flavivirus Immunogens

Various different technologies are currently used to develop flavivirus vaccines, such as live-attenuated vaccines, subunit, virus-like particles, inactivated, viral vector-based, epitope-based, DNA, and messenger RNA (mRNA) vaccines. However, despite the numerous advances that have been made in flavivirus vaccines, there is often a compromise between immunogenicity and efficacy. As a result, no highly effective and safe vaccines are currently available for preventing infection with flaviviruses, especially ZIKV, DENV, and WNV.

One of the biggest challenges for developing a highly effective and safe flavivirus vaccine is the ability to elicit potent neutralizing antibody responses that can cross-react to multiple flaviviruses without increasing the potential for ADE responses. ADE is a life-threatening phenomenon that is believed to have contributed to enhanced dengue disease observed in pediatric cohorts during the Dengvaxia® (CYD-TDV, Sanofi) clinical trials. The present disclosure is based, at least in part, on the surprising finding that, by replacing Domain II of the flavivirus envelope (E) protein, which is highly conserved among flaviviruses and one of the major targets of cross-reactive responses that lead to ADE, with flexible linkers, the resulting E protein subunit composed of only Domains I and III can fold into a soluble and well-expressed recombinant protein displaying key neutralizing epitopes for Zika and dengue neutralizing antibodies.

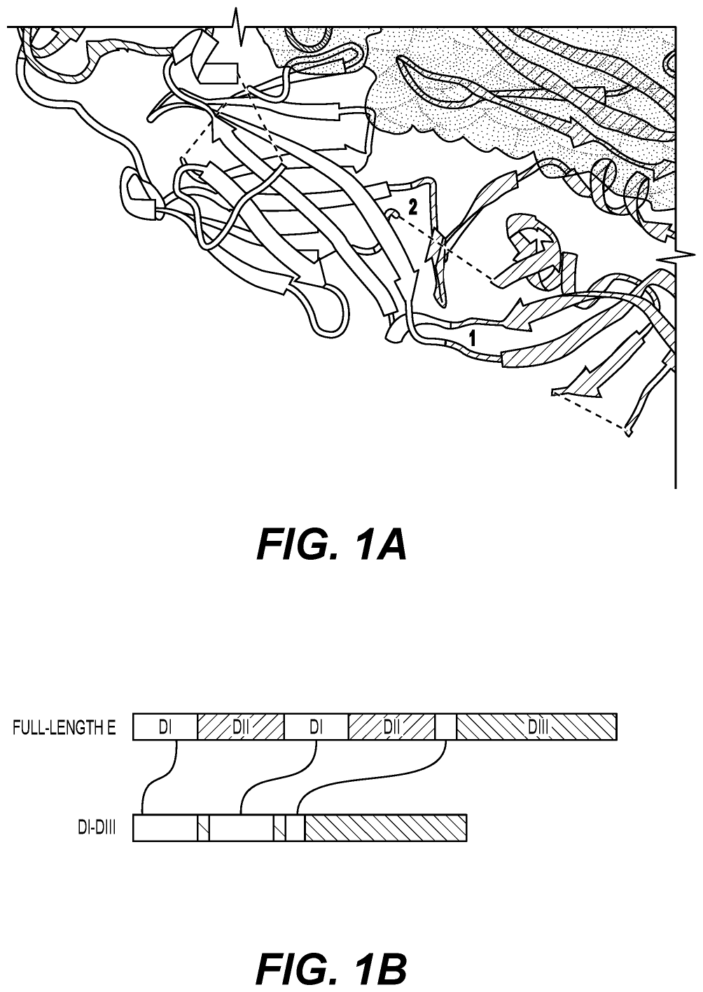

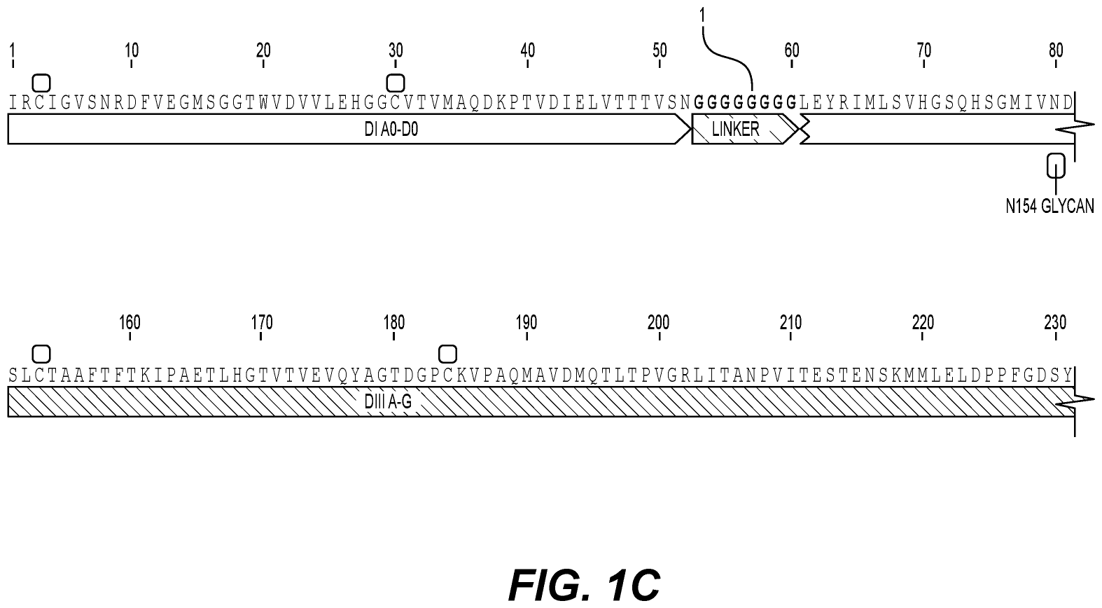

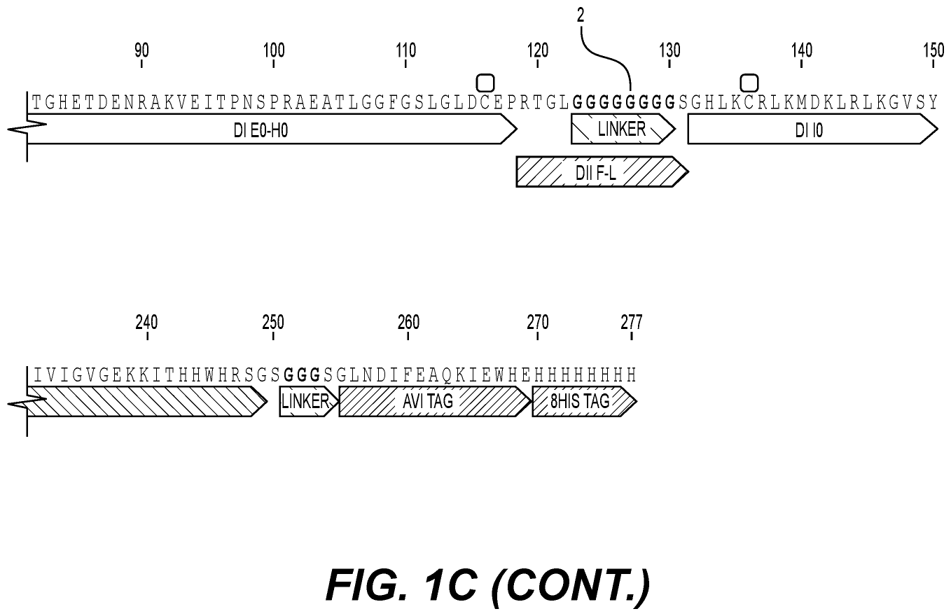

The flavivirus genome is translated as a single open reading frame (ORF) flanked by 5′ and 3′ untranslated regions. The ORF encodes a polyprotein that is cleaved by host and viral proteases into three structural proteins, the capsid (C) protein (105 amino acids (aa)), the premembrane/membrane (prM/M) protein (187 aa), and the envelope (E) protein (505 aa), as well as seven nonstructural (NS) proteins, NS1 (352 aa), NS2A (217 aa), NS2B (139 aa), NS3 (619 aa), NS4A (127 aa), NS4B (255 aa), and NS5 (904 aa). The E protein contains three structurally distinct domains, namely Domain I (DI), Domain II (DII), and Domain III (DIII). As shown in FIG. 1B (top), which is a schematic of the full-length flavivirus E protein, DI and DII are discontinuous peptides connected by four peptide linkers (not shown) to form the DI/DII hinge and DIII is a continuous peptide located at the C-terminus of the E protein and is connected by a flexible structure (not shown) to the opposite side of DI. This flexible structure connecting DI and DIII, also known as the DI-DIII linker, is a short polypeptide of 11 amino acids in length that is moderately conserved but exhibits poorly ordered structure in high-resolution crystal structures of the DENV serotype 2 soluble E prefusion dimer and postfusion trimer. DI contains 120 residues in three segments (residues 1-51, 137-189, and 285-302) and DIII contains approximately 100 amino acids. See e.g., Zhang et al., Viruses, 2017, 9:338, incorporated herein by reference.

DII of the flavivirus E protein is highly conserved and one of the major targets of cross-reactive responses that lead to ADE. By deleting DII, the immunogens of the disclosure preserve epitopes for potent DIII and DI-DIII linker monoclonal antibodies. Accordingly, the immunogens disclosed herein can be used as a prime and/or boost for flavivirus vaccination to target neutralization epitopes while minimizing ADE responses. Because each flavivirus E protein monomer is organized into three structurally distinct envelope domains (DI, DII, and DIII) and because DII is highly conserved, this strategy can also be utilized to engineer cross-protective DI-DIII immunogens from the E protein of other flaviviruses, such as DENV, which can then be used alone or in combination with DI-DIII immunogens of other flavivirus, such as ZIKV, to elicit cross-protective responses. Thus, in some embodiments, the immunogens of the disclosure can be designed for all flaviviruses, including, but not limited to, ZIKV, DENV serotypes 1 through 4, WNV, JEV, TBEV, and YFV, by removing DII from the E protein in a similar fashion. Vaccination strategies with DI-DIII immunogens from divergent flaviviruses, either at the same time or sequentially, would elicit broad cross neutralizing antibody responses against multiple flavivirus.

Accordingly, provided herein are immunogens comprising Formula (I):

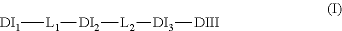

wherein DI₁, DI₂, and DI₃ together form Domain I (DI) of a flavivirus E protein, DIII is Domain III (DIII) of the same flavivirus E protein, and L₁ and L₂ are each independently a flexible linker, and wherein the immunogen does not comprise Domain II of the flavivirus E protein. In some embodiments, the flavivirus E protein is an E protein of Zika virus (ZIKV), dengue virus (DENV), Japanese Encephalitis Virus (JEV), West Nile Virus (WNV), Yellow Fever Virus (YFV), St Louis Encephalitis Virus (SLEV), Spondweni Virus (SPOV), Usutu Virus (USUV), Ilheus Virus (ILHV), Rocio Virus (ROCV), Wesselsbron Virus (WSLV), Tick Borne Encephalitis Virus (TBEV), Powassan Virus (POWV), Langat Virus (LGTV), Kyasanur Forest Disease (KSD), Alkhurma fever disease (AFD), or Omsk hemorrhagic fever virus (OHFV).

The sequences of DI and DIII of a flavivirus E protein suitable to form the immunogens of the disclosure can be determined based on the sequences of flaviviruses that are known and available in the public domain. Most of the existing flaviviruses have been sequenced and their complete genomic sequences, as well as the amino acid sequences of the encoded polyproteins, are available in the publicly accessible Genbank. For example, the complete genomic sequence of the Zika virus form a French polynesia outbreak in 2013 (strain H/PF/2013) is available in the GenBank database with Accession No. KJ776791.2. The polyprotein encoded by this Zika virus is also available in the GenBank database with Accession No. AHZ13508.1. Table 1 below provides some exemplary flaviviruses for which the complete genomic sequence and the amino acid sequence of the encoded polyprotein are known and available in the public domain.

TABLE 1
Accession numbers of the complete genomic sequence
and the amino acid sequence of the encoded polyprotein
of some exemplary flaviviruses.

| | Genomic | Encoded |
|---|---|---|
| Flavivirus | Sequence | Polyprotein |
| Zika virus strain H/PF/2013 | KJ776791.2 | AHZ13508.1 |
| Zika virus strain MR 766 | AY632535.2 | AAV34151.1 |
| Zika virus, strain MR 766-NIID | LC002520.1 | BAP47441.1 |
| Dengue virus 1 isolate MKS-0397 | KC762621.1 | AHG06305.1 |
| Dengue virus 2 strain ZS01/01 | KR920365.1 | AKU37073.1 |
| Dengue virus 3 strain CH53489 | DQ863638.1 | AAB69126.2 |
| Dengue virus type 4 strain DENV- | KJ579240.1 | AIQ84220.1 |
| 4/MT/BR2_TVP17888/2012 isolate | | |
| Spondweni virus strain SM-6 V-1 | DQ859064.1 | ABI54480.1 |
| West Nile virus strain NY99 | DQ211652.1 | ABA62343.1 |
| Kunjin virus isolate CH16078 | KX394384.1 | AOS89761.1 |
| Japanese encephalitis virus isolate 10S3 | MF542268.1 | ATQ62196.1 |
| Usutu virus | NC_006551.1 | YP_164264.1 |
| Murray Valley encephalitis virus | NC_000943.1 | NP_051124.1 |
| Murray Valley encephalitis virus strain | KM259934.1 | AIN35081.1 |
| 611W/WA/08 | | |
| Alfuy virus strain MRM3929 | AY898809.1 | AAX82481.1 |
| St. Louis encephalitis virus strain | FJ753286.2 | ACT31738.1 |
| CbaAr-4005 | | |
| Tembusu virus isolate FX2010 | MH414568.1 | AWV66902.1 |
| Tembusu virus isolate MM1775 | MH414569.1 | AWV66903.1 |
| Yellow fever virus isolate HD117294 | JX898868.1 | AFU76903.1 |
| from Senegal | | |
| Tick-borne encephalitis virus strain | KT001073.1 | ALA09091.1 |
| Lazo MP36 | | |

Taking the Zika virus strain H/PF/2013 as an example (polyprotein Accession No. AHZ13508), the E protein of this Zika virus has the following amino acid sequence with the amino acid sequence of DI in bold and the amino acid sequence of DIII italic:

(SEQ ID NO: 27)
IRCIGVSNRDFVEGMSGGTWVDVVLEHGGCVTVMAQDKPTVDIELVTTTV
SNMAEVRSYCYEASISDMASDSRCPTQGEAYLDKQSDTQYVCKRTLVDRG
WGNGCGLFGKGSLVTCAKFACSKKMTGKSIQPENLEYRIMLSVHGSQHSG
MIVNDTGHETDENRAKVEITPNSPRAEATLGGFGSLGLDCEPRTGLDFSD
LYYLTMNNKHWLVHKEWFHDIPLPWHAGADTGTPHWNNKEALVEFKDAHA
KRQTVVVLGSQEGAVHTALAGALEAEMDGAKGRLSSGHLKCRLKMDKLRL
KGVSYSLCTAAFTFTKIPAETLHGTVTVEVQYAGTDGPCKVPAQMAVDMQ
TLTPVGRLITANPVITESTENSKMMLELDPPFGDSYIVIGVGEKKITHHW
HRSGSTIGKAFEATVRGAKRMAVLGDTAWDFGSVGGALNSLGKGIHQIFG
AAFKSLFGGMSWFSQILIGTLLMWLGLNTKNGSISLMCLALGGVLIFLST
AVSAD

In some embodiments, the flavivirus E protein used to form the immunogens of the disclosure comprises an amino acid sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 27, including all values and subranges therebetween. In some embodiments, the flavivirus E protein used to form the immunogens of the disclosure comprises the amino acid sequence of SEQ ID NO: 27.

The amino acid sequences of DI₁, DI₂, DI₃, and DIII of the Zika virus strain H/PF/2013 E protein described herein, according to the present disclosure, are as follows:

DI₁:
(SEQ ID NO: 1)
IRCIGVSNRDFVEGMSGGTWVDVVLEHGGCVTVMAQDKPTVDIELVTTTV
SN
DI₂:
(SEQ ID NO: 2)
LEYRIMLSVHGSQHSGMIVNDTGHETDENRAKVEITPNSPRAEATLGGFG
SLGLDCEP
DI₃:
(SEQ ID NO: 3)
GHLKCRLKMDKLRLKGVSY
DIII:
(SEQ ID NO: 4)
SLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSG

Accordingly, in some embodiments, the immunogens of the disclosure comprise Formula (I):

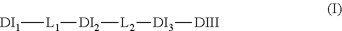

wherein DI₁ comprises an amino acid sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 1, including all values and subranges therebetween; DI₂ comprises an amino acid sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 2, including all values and subranges therebetween; DI₃ comprises an amino acid sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 3, including all values and subranges therebetween; and DIII comprises an amino acid sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 4, including all values and subranges therebetween; and wherein L₁ and L₂ each independently is a flexible linker. In some embodiments, the immunogens of the disclosure comprise Formula (I), wherein DII comprises an amino acid sequence at least about 90% identical to SEQ ID NO: 1, DI₂ comprises an amino acid sequence at least about 90% identical to SEQ ID NO: 2, DI₃ comprises an amino acid sequence at least about 90% identical to SEQ ID NO: 3, and DIII comprises an amino acid sequence at least about 90% identical to SEQ ID NO: 4. In some embodiments, the immunogens of the disclosure comprise Formula (I), wherein DI₁ comprises an amino acid sequence at least about 95% identical to SEQ ID NO: 1, DI₂ comprises an amino acid sequence at least about 95% identical to SEQ ID NO: 2, DI₃ comprises an amino acid sequence at least about 95% identical to SEQ ID NO: 3, and DIII comprises an amino acid sequence at least about 95% identical to SEQ ID NO: 4. In some embodiments, the immunogens of the disclosure comprise Formula (I), wherein DII comprises the amino acid sequence of SEQ ID NO: 1, DI₂ comprises the amino acid sequence of SEQ ID NO: 2; DI₃ comprises the amino acid sequence of SEQ ID NO: 3, and DIII comprises the amino acid sequence of SEQ ID NO: 4.

Any flexible linkers known in the art can be used to link DI₁, DI₂, and DI₃ to form the immunogens of the disclosure. In certain embodiments, the flexible linker has an amino acid sequence made up primarily of stretches of Gly and Ser residues (“GS” linker). Besides the GS linkers, many other flexible linkers have been designed for recombinant fusion proteins. It is understood that the flexible linker sequence is not a sequence that naturally occurs between DI₁, DI₂, and DI₃. In certain embodiments, the flexible linker has a length of 2, 3, 4, 5, 6, 7, 8, 9, 10, 11, 12, 13, 14, 15, 16, 17, 18, 19 or 20 amino acids, including all subranges therebetween. In certain embodiments, the linker peptide has a length of 4-8 amino acids. In certain embodiments, the linker peptide has a length of 9-20 amino acids.

In some embodiments, the flexible linker comprises glycine residues. In some embodiments, the flexible linker comprises at least 2, 3, 4, 5, 6, 7, 8, 9, or 10 glycine residues. In some embodiments, the flexible linker comprises 2, 3, 4, 5, 6, 7, 8, 9, or 10 consecutive glycine residues. Exemplary flexible linkers include, but are not limited to, the following:

| (SEQ ID NO: 5) | |
|---|---|
| | GGGGGGGG |
| (SEQ ID NO: 6) | |
| | GGGGGG |
| (SEQ ID NO: 7) | |
| | GGGG |
| (SEQ ID NO: 8) | |
| | GGGS |
| (SEQ ID NO: 9) | |
| | GGGGS |
| (SEQ ID NO: 10) | |
| | GGGGSGGGGS |
| (SEQ ID NO: 11) | |
| | GGGGSGGGGSGGGGS |
| (SEQ ID NO: 12) | |
| | GGGGSGGGGSGGGGSGGGGS |
| (SEQ ID NO: 13) | |
| | KESGSVSSEQLAQFRSLD |
| (SEQ ID NO: 14) | |
| | EGKSSGSGSESKST |
| (SEQ ID NO: 15) | |
| | GSAGSAAGSGEF |

Accordingly, in some embodiments, L₁ and L₂ each independently comprise one or more glycine residues and have a length of from about 4 to about 20 amino acids. In certain embodiments, L₁ and L₂ comprised in the immunogens of the disclosure each independently comprise the amino acid sequence of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

In some embodiments, L₁ comprised in the immunogens of the disclosure comprises the amino acid sequence of SEQ ID NO: 5. In some embodiments, L₁ comprised in the immunogens of the disclosure comprises the amino acid sequence of SEQ ID NO: 6. In some embodiments, L₁ comprised in the immunogens of the disclosure comprises the amino acid sequence of SEQ ID NO: 7. In some embodiments, L₁ comprised in the immunogens of the disclosure comprises the amino acid sequence of SEQ ID NO: 8. In some embodiments, L₁ comprised in the immunogens of the disclosure comprises the amino acid sequence of SEQ ID NO: 9. In some embodiments, L₁ comprised in the immunogens of the disclosure comprises the amino acid sequence of SEQ ID NO: 10. In some embodiments, L₁ comprised in the immunogens of the disclosure comprises the amino acid sequence of SEQ ID NO: 11. In some embodiments, L₁ comprised in the immunogens of the disclosure comprises the amino acid sequence of SEQ ID NO: 12. In some embodiments, L₁ comprised in the immunogens of the disclosure comprises the amino acid sequence of SEQ ID NO: 13. In some embodiments, L₁ comprised in the immunogens of the disclosure comprises the amino acid sequence of SEQ ID NO: 14. In some embodiments, L₁ comprised in the immunogens of the disclosure comprises the amino acid sequence of SEQ ID NO: 15.

In some embodiments, L₂ comprised in the immunogens of the disclosure comprises the amino acid sequence of SEQ ID NO: 5. In some embodiments, L₂ comprised in the immunogens of the disclosure comprises the amino acid sequence of SEQ ID NO: 6. In some embodiments, L₂ comprised in the immunogens of the disclosure comprises the amino acid sequence of SEQ ID NO: 7. In some embodiments, L₂ comprised in the immunogens of the disclosure comprises the amino acid sequence of SEQ ID NO: 8. In some embodiments, L₂ comprised in the immunogens of the disclosure comprises the amino acid sequence of SEQ ID NO: 9. In some embodiments, L₂ comprised in the immunogens of the disclosure comprises the amino acid sequence of SEQ ID NO: 10. In some embodiments, L₂ comprised in the immunogens of the disclosure comprises the amino acid sequence of SEQ ID NO: 11. In some embodiments, L₂ comprised in the immunogens of the disclosure comprises the amino acid sequence of SEQ ID NO: 12. In some embodiments, L₂ comprised in the immunogens of the disclosure comprises the amino acid sequence of SEQ ID NO: 13. In some embodiments, L₂ comprised in the immunogens of the disclosure comprises the amino acid sequence of SEQ ID NO: 14. In some embodiments, L₂ comprised in the immunogens of the disclosure comprises the amino acid sequence of SEQ ID NO: 15.

L₁ and L₂ can be different flexible linkers or identical to each other. In some embodiments, L₁ and L₂ are each independently selected from SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15, provided that L₁ and L₂ do not comprise the same amino acid sequence. In other embodiments, both L₁ and L₂ comprise the same amino acid sequence, such as the amino acid sequence of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

In addition to DII, DI₂, DI₃, DIII, L₁, and L₂, the immunogens of the disclosure can also comprise one or more heterologous peptides, such as a peptide tag, for example, an Avi tag (GLNDIFEAQKIEWHE; SEQ ID NO: 16) and/or a 8-His tag (HHHHHHHH; SEQ ID NO: 17), to facilitate purification and/or biotin labelling. In certain embodiments, the peptide tag is an Avi tag (GLNDIFEAQKIEWHE; SEQ ID NO: 16) or a 8-His tag (HHHHHHHH; SEQ ID NO: 17). Typically, the peptide tag is located at the C-terminus of the immunogen and may include an optional protease cleavage site, such as the human rhinovirus (HRV) 3C protease cleavage site (LEVLFQGP; SEQ ID NO: 18) or any other cleavage site known in the art. Additional flexible linkers as discussed herein can be used to link the one or more heterologous peptides to the C-terminus of the immunogens of the disclosure.

Accordingly, in some embodiments, the immunogens of the disclosure comprise an amino acid sequence at least about 40%, 45%, 50%, 55%, 60%, 65%, 70%, 75%, 80%, 85%, 95%, 96%, 97%, 98%, 99%, or 100% identical to the following amino acid sequence (flexible linkers in bold):

(SEQ ID NO: 19)
IRCIGVSNRDFVEGMSGGTWVDVVLEHGGCVTVMAQDKPTVDIELVTTTV
SNGGGGGGGGLEYRIMLSVHGSQHSGMIVNDTGHETDENRAKVEITPNSP
RAEATLGGFGSLGLDCEPRTGLGGGGGGGGSGHLKCRLKMDKLRLKGVSY
SLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSG.

In other embodiments, the immunogens of the disclosure comprise an amino acid sequence at least about 40%, 45%, 50%, 55%, 60%, 65%, 70%, 75%, 80%, 85%, 95%, 96%, 97%, 98%, 99%, or 100% identical to the following amino acid sequence (flexible linkers in bold, Avi tag in italic, and 8-His tag in bold and italic):

(SEQ ID NO: 20)
IRCIGVSNRDFVEGMSGGTWVDVVLEHGGCVTVMAQDKPTVDIELVTTTV
SNGGGGGGGGLEYRIMLSVHGSQHSGMIVNDTGHETDENRAKVEITPNSP
RAEATLGGFGSLGLDCEPRTGLGGGGGGGGSGHLKCRLKMDKLRLKGVSY
SLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSGS
GGGSGLNDIFEAQKIEWHEHHHHHHHH

In further embodiments, the immunogens of the disclosure comprise an amino acid sequence at least about 40%, 45%, 50%, 55%, 60%, 65%, 70%, 75%, 80%, 85%, 95%, 96%, 97%, 98%, 99%, or 100% identical to the following amino acid sequence (flexible linkers in bold, Avi tag in italic, 8-His tag in bold and italic, and HRV 3C protease cleavage site in bold and underlined):

(SEQ ID NO: 21)
IRCIGVSNRDFVEGMSGGTWVDVVLEHGGCVTVMAQDKPTVDIELVTTTV
SNGGGGGGGGLEYRIMLSVHGSQHSGMIVNDTGHETDENRAKVEITPNSP
RAEATLGGFGSLGLDCEPRTGLGGGGGGGGSGHLKCRLKMDKLRLKGVSY
SLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSGG
S [symbol] GGGSGLNDIFEAQKIEWHEHHHHHHHH

In some embodiments, the immunogens of the disclosure comprise the amino acid sequence of SEQ ID NO: 19. In some embodiments, the immunogens of the disclosure comprise the amino acid sequence of SEQ ID NO: 20. In some embodiments, the immunogens of the disclosure comprise the amino acid sequence of SEQ ID NO: 21.

In certain embodiments, the immunogens of the disclosure comprise an amino acid sequence at least about 40%, 45%, 50%, 55%, 60%, 65%, 70%, 75%, 80%, 85%, 95%, 96%, 97%, 98%, or 99% identical to SEQ ID NO: 19, SEQ ID NO: 20, or SEQ ID NO: 21, and have cross-reactivity with an anti-DIII antibody, such as an anti-ZIKV DIII antibody. In certain embodiments, the immunogens of the disclosure comprise an amino acid sequence at least about 40%, 45%, 50%, 55%, 60%, 65%, 70%, 75%, 80%, 85%, 95%, 96%, 97%, 98%, or 99% identical to SEQ ID NO: 19, SEQ ID NO: 20, or SEQ ID NO: 21, and have cross-reactivity with an anti-DI-DIII linker antibody, such as an anti-ZIKV DI-DIII linker antibody. In certain embodiments, the immunogens of the disclosure comprise an amino acid sequence at least about 40%, 45%, 50%, 55%, 60%, 65%, 70%, 75%, 80%, 85%, 95%, 96%, 97%, 98%, or 99% identical to SEQ ID NO: 19, SEQ ID NO: 20, or SEQ ID NO: 21, and have cross-reactivity with an anti-DIII antibody, such as an anti-ZIKV DIII antibody, and an anti-DI-DIII linker antibody, such as an anti-ZIKV DI-DIII linker antibody. Exemplary anti-DIII antibodies, such as anti-ZIKV DIII antibodies, include, but are not limited to, ZKA190 as disclosed in Stettler et al. (Science, 2016, 353 (6301): 823-826) and Z004 as disclosed in Robbiani et al. (Cell, 2017, 169 (4): 597-609 e11), both of which references are incorporated herein by reference. Exemplary anti-DI-DIII linker antibodies, such as anti-ZIKV DI-DIII linker antibodies, include, but are not limited to, MZ2 and MZ4 as disclosed in Dussupt et al. (Nature medicine, 2020, 26 (2): 228-235), incorporated herein by reference.

Also contemplated in the present disclosure are nucleic acid molecules that encode the immunogens disclosed herein. Such nucleic acid molecules can be used to produce the immunogens of the disclosure in a suitable expression system in vitro. In such embodiments, the nucleic acid molecules can comprise codon-optimized sequences to facilitate the expression of the encoded immunogens in the host cells. For instance, as exemplified in Example 1, the ZIKV DI-DIII immunogen can be expressed in the Drosophila S2 expression system using a codon-optimized sequence for expression in Drosophila melanogaster. In view of the recent development in DNA- and RNA-based vaccines, such nucleic acid molecules can also be used in vaccines to induce immune responses in a subject. In such embodiments, the nucleic acid molecules can comprise codon-optimized sequences to facilitate the expression of the encoded immunogens in the subject, such as a human. An exemplary codon-optimized sequence for expressing the ZIKV DI-DIII immunogen described in Example 1 may comprise the following sequence:

(SEQ ID NO: 23)
atcaggtgcatcggcgtgagcaatcgcgacttcgtggagggcatgtctgg
cggcacctgggggatgtggtgctggagcacggcggctgcgtgacagtgat
ggcccaggacaagccaaccgtggacatcgagctggtgaccacaaccgtgt
ccaacggcggaggcggaggcggaggcggactggagtacaggatcatgctg
agcgtgcacggcagccagcactccggcatgatcgtgaacgacacaggcca
cgagacagatgagaatagggccaaggtggagatcacacctaactccccaa
gggcagaggccaccctgggcggattcggctctctgggcctggactgcgag
cctaggacaggcctgggcggaggcggaggcggaggcggaagcggccacct
gaagtgccggctgaagatggataagctgagactgaagggcgtgtcctact
ctctgtgcacagccgccttcaccttcaccaagatccctgccgagacactg
cacggcacagtgaccgtggaggtgcagtatgccggcacagacggcccctg
taaggtgcctgcccagatggccgtggatatgcagacactgacccctgtgg
gccggctgatcaccgcaaatccagtgatcacagagtctaccgagaacagc
aagatgatgctggagctggacccccctttcggcgattcctatatcgtgat
cggcgtgggcgagaagaagatcacccaccactggcacagatccggaggcg
gatctggcctgaacgacatctttgaggcccagaagatcgagtggcacgag
caccatcaccatcaccatcaccattga.

Accordingly, in some embodiments, provided herein are nucleic acid molecules encoding any of the immunogens disclosed herein. The nucleic acid molecules can be DNA molecules in some embodiments, or RNA molecules in other embodiments. In some embodiments, the nucleic acid molecules of the disclosure are messenger RNA (mRNA) molecules. In some embodiments, the nucleic acid molecules of the disclosure comprise codon-optimized nucleic acid sequences.

In certain embodiments, the nucleic acid encodes the immunogen of SEQ ID NO: 19 and has the following nucleic acid sequence:

(SEQ ID NO: 24)
atcaggtgcatcggcgtgagcaatcgcgacttcgtggagggcatgtctgg
cggcacctgggtggatgtggtgctggagcacggcggctgcgtgacagtga
tggcccaggacaagccaaccgtggacatcgagctggtgaccacaaccgtg
tccaacggcggaggcggaggcggaggcggactggagtacaggatcatgct
gagcgtgcacggcagccagcactccggcatgatcgtgaacgacacaggcc
acgagacagatgagaatagggccaaggtggagatcacacctaactcccca
agggcagaggccaccctgggcggattcggctctctgggcctggactgcga
gcctaggacaggcctgggcggaggcggaggcggaggcggaagcggccacc
tgaagtgccggctgaagatggataagctgagactgaagggcgtgtcctac
tctctgtgcacagccgccttcaccttcaccaagatccctgccgagacact
gcacggcacagtgaccgtggaggtgcagtatgccggcacagacggcccct
gtaaggtgcctgcccagatggccgtggatatgcagacactgacccctgtg
ggccggctgatcaccgcaaatccagtgatcacagagtctaccgagaacag
caagatgatgctggagctggacccccctttcggcgattcctatatcgtga
tcggcgtgggcgagaagaagatcacccaccactggcacagatccgga.

In certain embodiments, the nucleic acid sequence encodes the immunogen of SEQ ID NO: 20 and has the following nucleic acid sequence:

(SEQ ID NO: 25)
atcaggtgcatcggcgtgagcaatcgcgacttcgtggagggcatgtctgg
cggcacctgggtggatgtggtgctggagcacggcggctgcgtgacagtga
tggcccaggacaagccaaccgtggacatcgagctggtgaccacaaccgtg
tccaacggcggaggcggaggcggaggcggactggagtacaggatcatgct
gagcgtgcacggcagccagcactccggcatgatcgtgaacgacacaggcc
acgagacagatgagaatagggccaaggtggagatcacacctaactcccca
agggcagaggccaccctgggcggattcggctctctgggcctggactgcga
gcctaggacaggcctgggcggaggcggaggcggaggcggaagcggccacc
tgaagtgccggctgaagatggataagctgagactgaagggcgtgtcctac
tctctgtgcacagccgccttcaccttcaccaagatccctgccgagacact
gcacggcacagtgaccgtggaggtgcagtatgccggcacagacggcccct
gtaaggtgcctgcccagatggccgtggatatgcagacactgacccctgtg
ggccggctgatcaccgcaaatccagtgatcacagagtctaccgagaacag
caagatgatgctggagctggacccccctttcggcgattcctatatcgtga
tcggcgtgggcgagaagaagatcacccaccactggcacagatccggatcc
ggaggoggatctggcctgaacgacatctttgaggcccagaagatcgagtg
gcacgagcaccatcaccatcaccatcaccattga

In certain embodiments, the nucleic acid sequence encodes the immunogen of SEQ ID NO: 21 and has the following nucleic acid sequence:

(SEQ ID NO: 26)
atcaggtgcatcggcgtgagcaatcgcgacttcgtggagggcatgtctgg
cggcacctgggtggatgtggtgctggagcacggcggctgcgtgacagtga
tggcccaggacaagccaaccgtggacatcgagctggtgaccacaaccgtg
tccaacggcggaggcggaggcggaggcggactggagtacaggatcatgct
gagcgtgcacggcagccagcactccggcatgatcgtgaacgacacaggcc
acgagacagatgagaatagggccaaggggagatcacacctaactccccaa
gggcagaggccaccctgggcggattcggctctctgggcctggactgcgag
cctaggacaggcctgggcggaggcggaggcggaggcggaagcggccacct
gaagtgccggctgaagatggataagctgagactgaagggcgtgtcctact
ctctgtgcacagccgccttcaccttcaccaagatccctgccgagacactg
cacggcacagtgaccgtggaggtgcagtatgccggcacagacggcccctg
taaggtgcctgcccagatggccgtggatatgcagacactgacccctgtgg
gccggctgatcaccgcaaatccagtgatcacagagtctaccgagaacagc
aagatgatgctggagctggacccccctttcggcgattcctatatcgtgat
cggcgtgggcgagaagaagatcacccaccactggcacagatccggaggat
ccctggaggtgctgttccagggcccaggaggcggatctggcctgaacgac
atctttgaggcccagaagatcgagtggcacgagcaccatcaccatcacca
tcaccattga.

In some embodiments, the nucleic acid molecules of the disclosure comprise a nucleotide sequence or a ribonucleotide sequence at least about 40%, 45%, 50%, 55%, 60%, 65%, 70%, 75%, 80%, 85%, 90%, 91%, 92%, 93%, 94%, 95%, 96%, 97%, 98%, 99%, or 100% identical to SEQ ID NO: 22, SEQ ID NO: 23, SEQ ID NO: 24, SEQ ID NO: 25, or SEQ ID NO: 26. In some embodiments, the nucleic acid molecules of the disclosure comprise a nucleotide sequence or a ribonucleotide sequence that is 100% identical to SEQ ID NO: 22, SEQ ID NO: 23, SEQ ID NO: 24, SEQ ID NO: 25, or SEQ ID NO: 26.

In certain embodiments, the nucleic acid molecules of the disclosure comprise a nucleotide sequence or a ribonucleotide sequence at least about 40%, 45%, 50%, 55%, 60%, 65%, 70%, 75%, 80%, 85%, 90%, 91%, 92%, 93%, 94%, 95%, 96%, 97%, 98%, or 99% identical to SEQ ID NO: 22, SEQ ID NO: 23, SEQ ID NO: 24, SEQ ID NO: 25, or SEQ ID NO: 26 and encode a polypeptide having cross-reactivity with an anti-DIII antibody, such as an anti-ZIKV DIII antibody. In certain embodiments, the nucleic acid molecules of the disclosure comprise a nucleotide sequence or a ribonucleotide sequence at least about 40%, 45%, 50%, 55%, 60%, 65%, 70%, 75%, 80%, 85%, 90%, 91%, 92%, 93%, 94%, 95%, 96%, 97%, 98%, or 99% identical to SEQ ID NO: 22, SEQ ID NO: 23, SEQ ID NO: 24, SEQ ID NO: 25, or SEQ ID NO: 26 and encode a polypeptide having cross-reactivity with an anti-DI-DIII linker antibody, such as an anti-ZIKV DI-DIII linker antibody. In certain embodiments, the nucleic acid molecules of the disclosure comprise a nucleotide sequence or a ribonucleotide sequence at least about 40%, 45%, 50%, 55%, 60%, 65%, 70%, 75%, 80%, 85%, 90%, 91%, 92%, 93%, 94%, 95%, 96%, 97%, 98%, or 99% identical to SEQ ID NO: 22, SEQ ID NO: 23, SEQ ID NO: 24, SEQ ID NO: 25, or SEQ ID NO: 26 and encode a polypeptide having cross-reactivity with an anti-DIII antibody, such as an anti-ZIKV DIII antibody, and an anti-DI-DIII linker antibody, such as an anti-ZIKV DI-DIII linker antibody.

Due to their small size (about 30 kDa), the immunogens of the disclosure can also be multimerized on nanoparticle scaffolds, such as Helicobacter pylori ferritin-based nanoparticles, allowing for improved immunogenicity via display of, for instance, 24-mers. Other multimerizing proteins, such as lumazine synthase or other engineered and synthetic scaffolds, can also be used.

In some embodiments, such nanoparticles can be homotypic, i.e., present only DI-DIII from a single flavivirus, such as ZIKV. In some embodiments, mosaic nanoparticles can be made, displaying DI-DIII from, for example, ZIKV and strains of DENV, or other flaviviruses, to elicit broad antibody responses across multiple flaviviruses.

### Compositions and Vaccines

Also provided herein are compositions comprising any of the immunogens disclosed herein or the nucleic acid molecules, such as DNA molecules or RNA molecules (e.g., mRNA molecules) encoding the immunogens disclosed herein. In some embodiments, the compositions of the disclosure may further comprise one or more carriers, targeting ligands, stabilizing reagents (e.g., preservatives and antioxidants), and/or other pharmaceutically acceptable excipients to stabilize the DNA or RNA molecules (e.g., to prolong the shelf-life of the composition), to facilitate administration of the composition, and/or to enhance in vivo expression of the DNA or RNA molecules. Examples of such excipients include, but are not limited to, parabens, thimerosal, thiomersal, chlorobutanol, bezalkonium chloride, and chelators (e.g., ethylenediaminetetraacetic acid, or EDTA).

The compositions of the disclosure can be formulated for administration in any way known in the art of drug delivery, for example, orally, parenterally, intravenously, intramuscularly, subcutaneously, intradermally, transdermally, intrathecally, submucosally, sublingually, rectally, vaginally, etc. In some embodiments, the composition is formulated for sublingual administration, intramuscular administration, intradermal administration, subcutaneous administration, intravenous administration, intranasal administration, administration by inhalation, or intraperitoneal administration. In some embodiments, the composition is formulated for sublingual administration. In some embodiments, the composition is formulated for intramuscular injection.

In some embodiments, the compositions of the disclosure are immunogenic compositions. As used herein, the term “immunogenic composition” refers to a composition that generates an immune response that may or may not be a protective immune response or protective immunity. The term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Also provided herein is a vaccine comprising the immunogenic composition of the disclosure and a pharmaceutically acceptable carrier. As used herein, the term “vaccine” refers to a composition that generates a protective immune response or a protective immunity in a subject. A “protective immune response” or “protective immunity” refers to an immune response that protects a subject from infection (prevents infection or prevents the development of disease associated with infection) or reduces the symptoms of infection (for instance, an infection by a flavivirus virus). Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, intranasal, by inhalation, or intramuscular.

In some embodiments, the immunogenic composition of the disclosure comprises an adjuvant. In other embodiments, the immunogenic composition of the disclosure does not contain an adjuvant. Similarly, in some embodiments, the vaccine of the disclosure can be administered with an adjuvant to boost the immune response. In other embodiments, the vaccines can be administered without an adjuvant. As used herein, the term “adjuvant” refers to a substance or combination of substances that may be used to enhance an immune response to an antigen component of a vaccine or immunogenic composition. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include, but are not limited to, AS04 (Didierlaurent et al., J. Immunol., 2009, 183:6186-6197), IL-2, RANTES, GM-CSF, TNF-α, IFN-γ, G-CSF, LFA-3, CD72, B7-1, B7-2, OX-40L and 41 BBL.

In some embodiments, the immunogenic composition or vaccine of the disclosure is formulated for parenteral administration, such as intravenous, subcutaneous, intraperitoneal, intradermal, or intramuscular. The immunogenic composition or vaccine of the disclosure may also be formulated for intranasal or inhalation administration. The immunogenic composition or vaccine of the disclosure can also be formulated for any other intended route of administration.

In some embodiments, the immunogenic composition or vaccine of the disclosure is formulated for intradermal injection, intranasal administration or intramuscular injection. General considerations in the formulation and manufacture of pharmaceutical agents for administration by these routes may be found, for example, in Remington's Pharmaceutical Sciences, 19th ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the immunogenic composition or vaccine of the disclosure is administered using a device that delivers a metered dosage of the vaccine composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662, all of which are incorporated herein by reference. Intradermal compositions may also be administered by devices which limit the effective penetration length of a needle into the skin, such as those described in WO1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537, all of which are incorporated herein by reference. Additionally, conventional syringes may be used in the classical Mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

### Methods of Use

Also provided herein are methods of administering the vaccines described herein to a subject. The methods may be used to vaccinate a subject to prevent a flavivirus infection in the subject, to decrease the subject's likelihood of getting a flavivirus infection, or to reduce the subject's likelihood of getting serious illness from a flavivirus infection, including, but not limited to, ZIKV infection or DENV infection. Likewise, the present disclosure provides any of the vaccine compositions described herein for use in vaccinating a subject against a flavivirus infection. Also disclosed is any of the vaccine compositions as described herein, for the manufacture of a vaccine for use in vaccinating a subject against a flavivirus infection. In some embodiments, the vaccination method or use comprises administering to a subject in need thereof an immunologically effective amount of any of the vaccines described herein.

As used herein, the term “immunologically effective amount” or “therapeutically effective amount” means an amount sufficient to immunize a subject. In some embodiments, the immunologically effective amount or therapeutically effective amount is capable of eliciting protective immunity against a flavivirus, including, but not limited to, ZIKV or DENV, which include, but are not limited to, an increase of antibody titers and/or T cell immunity against a flavivirus, including, but not limited to, ZIKV or DENV. In some embodiments, an immunologically effective amount or therapeutically effective amount of the vaccine or composition as disclosed herein increases protective immunity in a subject by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including values and subranges therebetween, when compared with a subject who is not administered with the vaccine or composition as disclosed herein.

Accordingly, in some embodiments, the disclosure provides a method of immunizing a subject comprising administering to the subject in need thereof an immunologically effective amount of any of the vaccines described herein. As used herein, “immunize” or “immunizing” means to induce in a subject a protective immune response against a flavivirus infection. Likewise, the present disclosure provides any of the vaccine compositions described herein for use in immunizing a subject against a flavivirus infection, including, but not limited to, ZIKV infection or DENV infection. Also disclosed is any of the vaccine compositions as described herein, for the manufacture of a vaccine for use in immunizing a subject against a flavivirus infection, including, but not limited to, ZIKV infection or DENV infection.

In some embodiments, the method or use prevents flavivirus infection or disease caused by the flavivirus infection in the subject, including, but not limited to, ZIKV infection or DENV infection. In some embodiments, the method or use decreases the subject's likelihood of getting a flavivirus infection by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including values and subranges therebetween, when compared with a subject who is not administered with the vaccine or composition as disclosed herein. In some embodiments, the method or use reduces the subject's likelihood of getting serious illness from a flavivirus infection, including, but not limited to, ZIKV infection or DENV infection, by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including values and subranges therebetween, when compared with a subject who is not administered with the vaccine or composition as disclosed herein. In some embodiments, the method or use raises a protective immune response in the subject. In some embodiments, the protective immune response is an antibody response.

Also provided, in some embodiments, is a method of reducing one or more symptoms of a flavivirus infection, including, but not limited to, ZIKV infection or DENV infection, comprising administering to a subject in need thereof a prophylactically effective amount of any of the vaccines described herein. Likewise, the present disclosure provides any of the vaccine compositions described herein for use in (or for the manufacture of a medicament for use in) reducing one or more symptoms of a flavivirus infection, including, but not limited to, ZIKV infection or DENV infection. Also disclosed is any of the vaccine compositions as described herein, for the manufacture of a vaccine for use in reducing one or more symptoms of a flavivirus infection, including, but not limited to, ZIKV infection or DENV infection, in a subject. In some embodiments, the method or use of the present disclosure reduces one or more symptoms of a flavivirus infection, including, but not limited to, ZIKV infection or DENV infection, by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including values and subranges therebetween, when compared with a subject who is not administered with the vaccine or composition as disclosed herein.

In such embodiments, the vaccine, and an optional adjuvant, may be administered prior to or after development of one or more symptoms of the flavivirus infection. That is, in some embodiments, the vaccines described herein may be administered prophylactically to prevent the flavivirus infection or ameliorate the symptoms of a potential flavivirus infection, including, but not limited to, ZIKV infection or DENV infection.

In some embodiments, the subject is at risk of infection if the subject will be in contact with other individuals or livestock (e.g., swine) known or suspected to have been infected with a flavivirus and/or if the subject will be present in a location in which flavivirus infection, including, but not limited to, ZIKV infection or DENV infection, is known or thought to be prevalent or endemic. In some embodiments, the vaccines are administered to a subject suffering from a flavivirus infection, including, but not limited to, ZIKV infection or DENV infection, or the subject is displaying one or more symptoms commonly associated with a flavivirus infection, including, but not limited to, ZIKV infection or DENV infection. In some embodiments, the subject is known or believed to have been exposed to a flavivirus infection, including, but not limited to, ZIKV infection or DENV infection.

Vaccines in accordance with the disclosure may be administered in any amount or dose appropriate to achieve a desired outcome. In some embodiments, the desired outcome is induction of a lasting adaptive immune response against the flavivirus. In some embodiments, the desired outcome is reduction in intensity, severity, and/or frequency, and/or delay of onset of one or more symptoms associated with flavivirus infection. The dose required may vary from subject to subject depending on the species, age, weight and general condition of the subject, the severity of the infection being treated, the particular composition being used, and its mode of administration.

In some embodiments, the vaccines described herein are administered to subjects, wherein the subjects can be any member of the animal kingdom. In some embodiments, the subject is a non-human animal. In some embodiments, the non-human subject is an avian (e.g., a chicken or a bird), a reptile, an amphibian, a fish, an insect, and/or a worm. In some embodiments, the non-human subject is a mammal (e.g., a ferret, a rodent, a mouse, a rat, a rabbit, a monkey, a dog, a cat, a sheep, cattle, a primate, and/or a pig).

In some embodiments, the vaccines described herein are administered to a human subject. In some embodiments, a human subject is 6 months of age or older, 6 months through 35 months of age, at least two years of age, at least 3 years of age, 36 months through 8 years of age, 9 years of age or older, at least 6 months of age and less than 5 years of age, at least 6 months of age and less than 18 years of age, or at least 3 years of age and less than 18 years of age. In some embodiments, the human subject is an infant (less than 36 months). In some embodiments, the human subject is a child or adolescent (less than 18 years of age). In some embodiments, the human subject is a child of at least 6 months of age and less than 5 years of age. In some embodiments, the human subject is at least 5 years of age and less than 60 years of age. In some embodiments, the human subject is at least 5 years of age and less than 65 years of age. In some embodiments, the human subject is elderly (at least 60 years of age or at least 65 years of age). In some embodiments, the human subject is a non-elderly adult (at least 18 years of age and less than 65 years of age or at least 18 years of age and less than 60 years of age).

The methods and uses of the vaccines described herein include administration of a single dose to a subject (i.e., no booster dose). In some embodiments, the methods and uses of the vaccines described herein include prime-boost vaccination strategies. Prime-boost vaccination comprises administering a priming vaccine and then, after a period of time has passed, administering to the subject a boosting vaccine. The immune response is “primed” upon administration of the priming vaccine and is “boosted” upon administration of the boosting vaccine. The priming vaccine can include a vaccine as described herein and an optional adjuvant. Likewise, the boosting vaccine can include a vaccine as described herein and an optional adjuvant. The priming vaccine can be, but need not be, the same as the boosting vaccine. Administration of the boosting vaccine is generally weeks or months after administration of the priming composition, preferably about 2-3 weeks or 4 weeks, or 8 weeks, or 16 weeks, or 20 weeks, or 24 weeks, or 28 weeks, or 32 weeks. In certain embodiments, the recipient of the prime-boost vaccination is a naïve subject, typically a naïve infant or child.

The vaccine can be administered using any suitable route of administration, including, for example, parenteral delivery, as discussed above. In some embodiments, the vaccine is administered intramuscularly, intradermally, subcutaneously, intravenously, intranasally, by inhalation, or intraperitoneally.

### Other Methods

Also provided herein is a method of identifying an antibody against flavivirus in a sample, the method comprising: a) contacting a sample with at least one polypeptide comprising an amino acid sequence at least about 95%, 96%, 97%, 98%, 99%, or 100% identical to the amino acid sequence of SEQ ID NO: 19, SEQ ID NO: 20, or SEQ ID NO: 21; and b) determining formation of a complex between the at least one polypeptide and at least one substance in the sample, wherein formation of the complex indicates that the at least one substance is an antibody against flavivirus. Further provided is a method of identifying a B cell lymphocyte expressing an antibody that binds to an antigen of a flavivirus, the method comprising: a) contacting a B cell lymphocyte in a sample with at least one polypeptide comprising an amino acid sequence at least about 95%, 96%, 97%, 98%, 99%, or 100% identical to the amino acid sequence of SEQ ID NO: 19, SEQ ID NO: 20, or SEQ ID NO: 21; and b) determining formation of a complex between the B cell lymphocyte and the at least one polypeptide, wherein formation of the complex indicates that the B cell lymphocyte expresses an antibody that binds to an envelope (E) protein of the flavivirus. In some embodiments, the at least one polypeptide is labeled with one or more detectable labels, as known in the art, and the complex is detected. In certain embodiments, the label is one or more compounds that are able to emit fluorescence and the complex is determined using a fluorescence-activated cell sorting device. Any fluorescence-activated cell sorting devices known in the art can be used. In other embodiments, the at least one polypeptide is labeled with one or more chemicals that are able to emit chemiluminescent light, and the complex is determined using a device that is capable of detecting chemiluminescent light. Any device that is capable of detecting chemiluminescent light known in the art can be used. In some embodiments, the at least one polypeptide used in any of the methods disclosed herein comprises the amino acid sequence of SEQ ID NO: 19, SEQ ID NO: 20, or SEQ ID NO: 21.

Any sample can be used in the methods disclosed herein. In some embodiments, the sample is a tissue sample or a body fluid sample. In some embodiments, the sample is a body fluid sample comprising blood, plasma, serum, saliva, tear, urine, cerebrospinal fluid, pleural effusion, ascites, or peritoneal effusion.

The methods disclosed here can be used to identify an antibody against any flavivirus, or a B cell lymphocyte expressing an antibody that binds to an antigen of any flavivirus, including, but not limited to, Zika virus (ZIKV), dengue virus (DENV), Japanese Encephalitis Virus (JEV), West Nile Virus (WNV), Yellow Fever Virus (YFV), St Louis Encephalitis Virus (SLEV), Spondweni Virus (SPOV), Usutu Virus (USUV), Ilheus Virus (ILHV), Rocio Virus (ROCV), Wesselsbron Virus (WSLV), Tick Borne Encephalitis Virus (TBEV), Powassan Virus (POWV), Langat Virus (LGTV), Kyasanur Forest Disease (KSD), Alkhurma fever disease (AFD), and/or Omsk hemorrhagic fever virus (OHFV).

### EXAMPLES

The following examples are to be considered illustrative and not limiting on the scope of the disclosure described above.

### Example 1. Design, Construction, Expression, and Characterization of a ZIKV DI-DIII Immunogen

A novel conformational epitope on the ZIKV E protein that spans Domains I and III (DI and DIII, respectively) and the DI-DIII linker targeted by cross-neutralizing antibodies to multiple flaviviruses was previously identified (Dussupt et al., Nat. Med., 2020, 26 (2): 228-235). Domain II (DII) of the flavivirus E protein is highly conserved and one of the major targets of cross-reactive responses that lead to ADE. Thus, in this study, a novel ZIKV E immunogen, devoid of DII but retaining DI and DIII, was designed by engineering the ZIKV E protein and replacing DII with flexible linkers to retain the native folding of DI, leading to a subunit of the ZIKV E protein called DI-DIII. DI-DIII remained soluble, resulting in an efficiently secreted recombinant protein subunit displaying key neutralizing epitopes for ZIKV and DENV neutralizing antibodies. DI-DIII can be used as an immunogen in a vaccine for ZIKV and flavivirus vaccination to target neutralization epitopes devoid of ADE responses. This strategy can be utilized to engineer DI-DIII immunogens not only for ZIKV, as demonstrated in this Example, but also using other flavivirus E proteins, which are similarly organized into three structurally distinct domains (DI, DII, and DIII), such as DENV, JEV, YFV, and WNV.

### 1. Methods

The ZIKV DI-DIII construct was designed and engineered from available structural information on the ZIKV E protein (PDB 5LBV; pdbj.org/emnavi/quick.php?id=pdb-5lbv) and cloned into the inducible pMT-BiP vector (ThermoFisher) as part of the Drosophila S2 expression system (ThermoFisher). Briefly, the inducible pMT-BiP vector (ThermoFisher) containing the full-length ZIKV (PRVABC59) E sequence codon optimized for Drosophila melanogaster with a BiP signal sequence and under the inducible metallothionein promoter as well as with C-terminal Avi and His tags was modified to replace the DII-coding sequence with a nucleotide sequence encoding two 8-glycine linkers using site-directed mutagenesis. The resulting DI-DIII construct encodes the following amino acid sequence with the two 8-glycine linkers shown in bold and the C-terminal Avi and His tags underlined:

(SEQ ID NO: 20)
IRCIGVSNRDFVEGMSGGTWVDVVLEHGGCVTVMAQDKPTVDIELVTTTV
SNGGGGGGGGLEYRIMLSVHGSQHSGMIVNDTGHETDENRAKVEITPNSP
RAEATLGGFGSLGLDCEPRTGLGGGGGGGGSGHLKCRLKMDKLRLKGVSY
SLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSGG
GGSGLNDIFEAQKIEWHEHHHHHHHH

The Drosophila codon optimized sequence encoding this DI-DIII construct has the following sequence:

(SEQ ID NO: 22)
atccgctgcatcggcgtgtccaaccgcgacttcgtggagggcatgagcgg
cggcacctgggtggatgtggtgctggagcacggtggctgcgtgaccgtga
tggcccaggacaagcccaccgtggatatcgagctggtgaccaccaccgtg
tccaacggaggcggcggtggcggaggcggtctggagtaccgcatcatgct
gtccgtgcacggctcccagcatagcggcatgatcgtgaacgacaccggcc
acgagaccgatgagaaccgcgccaaggtggagatcacccccaacagcccc
cgcgccgaggccaccctgggcggcttcggcagcctgggcctggactgcga
gccccgcaccggcctgggcggcggtggaggcggaggtggcagcggccatc
tgaagtgccgcctgaagatggataagctgcgcctgaagggcgtgtcctac
agcctgtgcaccgccgccttcaccttcaccaagattcccgccgagaccct
gcatggcaccgtgaccgtggaggtgcagtacgccggcaccgatggcccct
gcaaggtgcccgcccagatggccgtggatatgcagaccctgacccccgtg
ggccgcctgatcaccgccaaccccgtgatcaccgagtccaccgagaacag
caagatgatgctggagctggacccgcccttcggcgattcctacatcgtga
tcggcgtgggcgagaagaagatcacccaccattggcatcgctccggctcc
ggcggcggcagcggcctgaacgatatcttcgaggcccagaagatcgagtg
gcatgagcaccatcatcaccaccatcaccattga

Stably transfected cell lines were generated upon blasticidin selection and adapted to serum-free conditions. Expression was induced with 0.5 mM CuSO₄ and supernatant was harvested 7 days later. DI-DIII was purified from cell culture supernatant using Ni-NTA (Qiagen) affinity chromatography followed by size exclusion chromatography using an Enrich SEC650 column (Bio-Rad) on a NGC fast protein liquid chromatography system (Bio-Rad).

For binding assessment by biolayer chromatography on an Octet RED96 instrument (ForteBio), DI-DIII was biotinylated using the BirA biotinylation kit (Avidity) and immobilized to streptavidin sensors. After establishing baseline in kinetic buffer (ForteBio), DI-DIII was dipped into well containing characterized monoclonal antibodies at 200 nM for 450 seconds.

For assessment of vaccinated donor B cells expressing B cell receptors (BCRs) capable of recognizing DI-DIII, biotinylated DI-DIII or full-length ZIKV E protein was tetramerized (4:1 molar ratio) with streptavidin-fluorophore conjugates BUV737 (BioLegend) or PE (ThermoFisher), respectively. Cryopreserved peripheral blood mononuclear cells (PBMCs) from two ZIKV vaccinated donors and one control donor were thawed in warm medium containing benzonase, then washed with PBS and stained for viability using Invitrogen Aqua Live/Dead stain. Cells were incubated at 4° C. for 30 minutes with a cocktail of antibodies including CD3 BV510 (BD Biosciences), CD4 BV510 (BD Biosciences), CD8 BV510 (BioLegend), CD14 BV510 (BioLegend), CD16 BV510 (BD Biosciences), and CD56 BV510 (BioLegend) as dump channel markers, and CD19 PE Dazzle 594 (BioLegend), IgG BUV496 (BD Biosciences), IgD APC-H7 (BD Biosciences), IgA PerCP-Vio700 (Milteni), IgM BUV395 (BD Biosciences), CD20 AF700 (BD Biosciences), Integrin B7 PE-Cy5 (BD Biosciences), CD38 PE-Cy7 (BioLegend), CD21 FITC (BioLegend), CD27 BV605 (BioLegend), and CD10 BV650 (BD Biosciences). ZIKV E PE and DI-DIII BUV737 tetramers were included in the antibody cocktail. Single, antigen-specific B cells (CD19⁺/IgD⁻ and either single or double ZIKV E/DI-DIII positive) were sorted into 96-well plates using a FACSAria (Becton Dickinson).

### 2. Results

i. Removing DII, the Primary Target of FLE Responses

As demonstrated herein, removal of DII that contains the fusion loop epitope (FLE) can be used to prevent the elicitation of antibody responses targeting the FLE, which are highly cross-reactive across flaviviruses but often poorly neutralizing, causing risks of ADE of infection. DI-DIII presents potent neutralizing epitopes in DIII and in the DI-DIII linker, a novel epitope previously characterized when MZ4, a potent ZIKV and DENV-2 neutralizing antibody, was isolated (Dussupt et al., Nat. Med., 2020, 26 (2): 228-235). While DI and DII are two distinct domains of the ZIKV E protein, the polypeptide chain goes back and forth twice across those two domains (FIGS. 1A and 1B). The ZIKV E protein was engineered by excising DII and adding two flexible synthetic linkers, each consisting of 8 glycines, to piece together the three parts that constitute DI and allow DI to adopt its normal fold in the absence of DII (FIG. 1C).

ii. The Recombinant DI-DIII Engineered Subunit is Recombinantly Expressed at High Yield

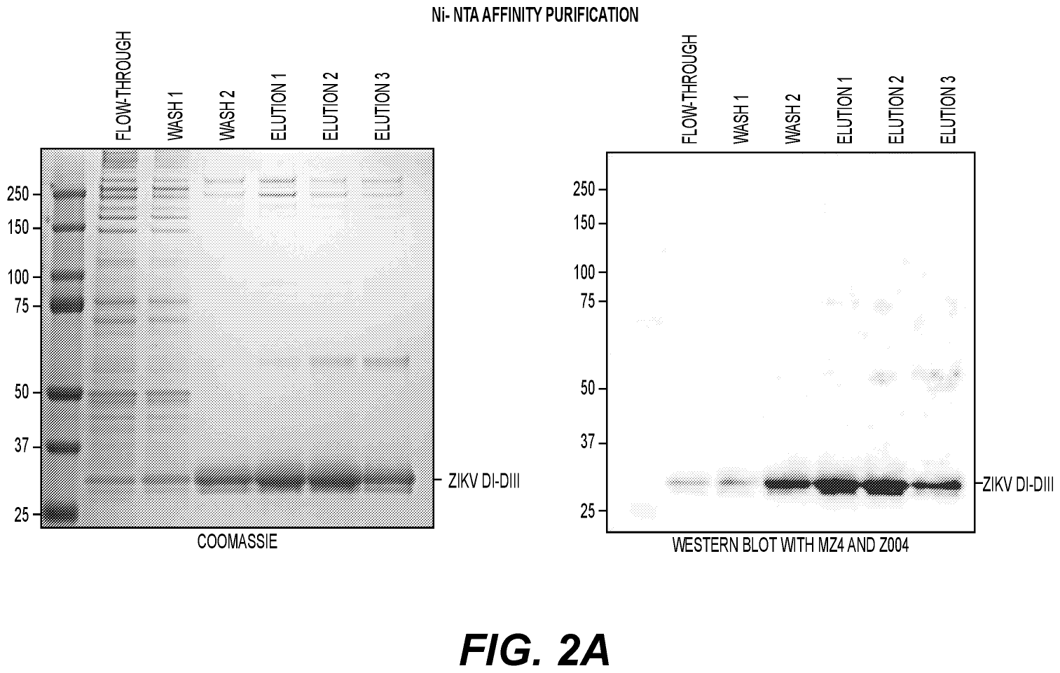

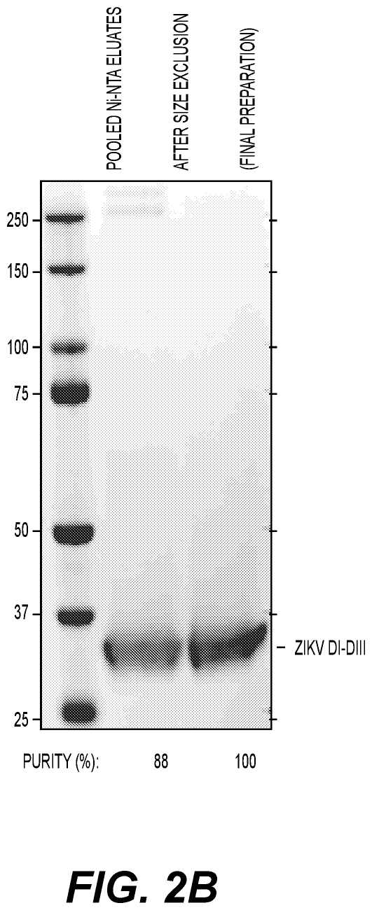

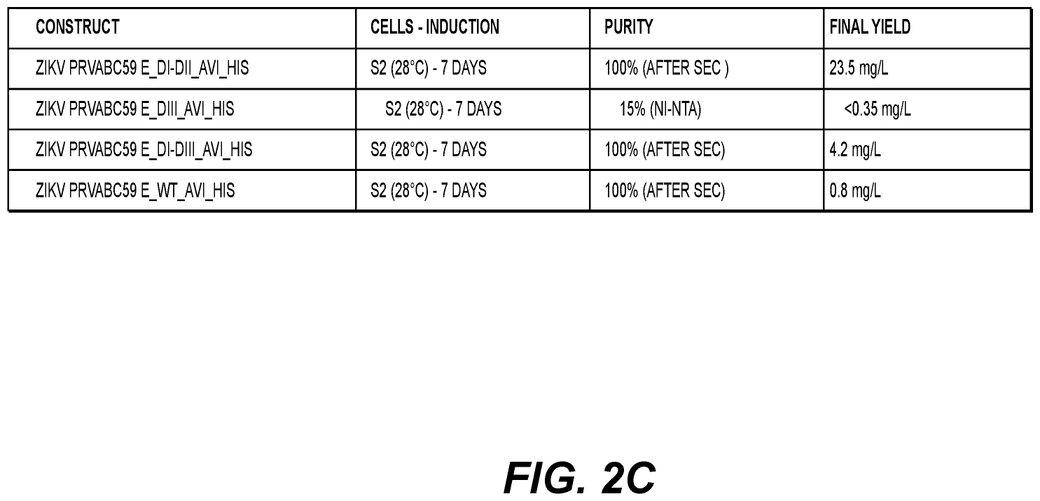

The expression of DI-DIII in the Drosophila S2 cell system, commonly used for expression of flavivirus proteins, was tested. DI-DIII was purified from stably transfected S2 cell culture supernatant using Ni-NTA affinity columns and eluted at about 88% purity (FIG. 2A, left panel). Identity was confirmed by western blot using DIII and DI-DIII reactive antibodies, which both bound to the eluted product (FIG. 2A, right panel). A size exclusion chromatography polishing step improved purity further to 100% (FIG. 2B). In addition to DI-DIII, other fragments of the ZIKV E protein, such as DI-DII and DIII, as well as the full-length wild-type ZIK V E protein, were also expressed and purified using the same methods. Both purity and yield were evaluated and it was observed that DI-DIII had about 5-fold improved yield over the full-length E protein (FIG. 2C). DIII alone could not even be produced in this system, as purity after the Ni-NTA affinity step was too low, suggesting a poor expression or secretion of this subunit. DIII has been explored as a potential immunogen in multiple vaccination strategies as it contains epitopes that bind to broad neutralizing antibodies. Compared to DIII, DI-DIII appears to be a better choice for subunit vaccine development due to its vastly increased yield (FIG. 2C) and the fact that it presents additional neutralizing epitopes, such as the DI-DIII linker and possibly some other epitopes in DI.

iii. Confirmation of DI-DIII Antigenic Properties

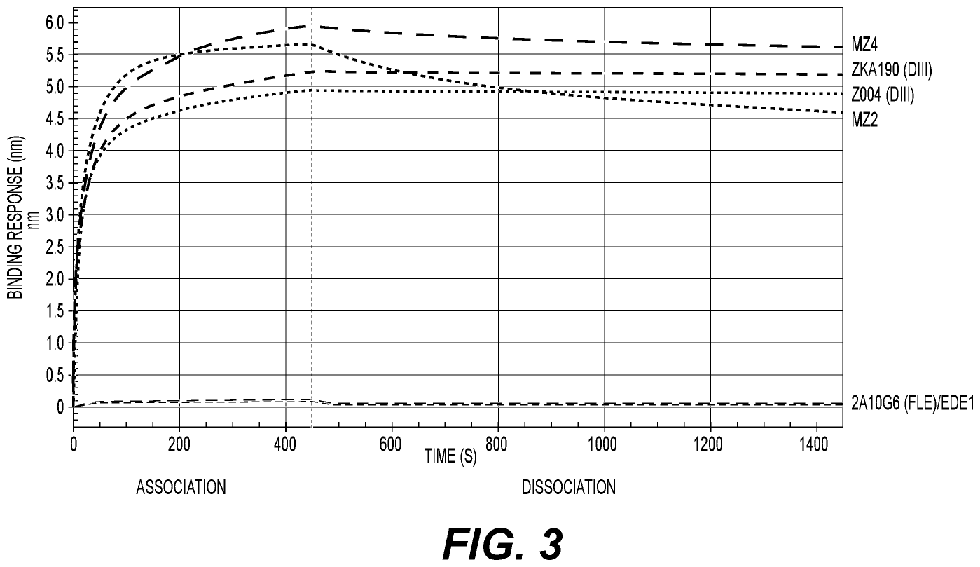

The produced DI-DIII protein was then tested against a panel of characterized monoclonal antibodies, recognizing DIII, the DI-DIII linker and the FLE using biolayer interferometry (BLI). DIII mAbs (ZKA190 (Stettler et al., Science, 2016, 353 (6301): 823-826, Z004 (Robbiani et al., Cell, 2017, 169 (4): 597-609 e11)) bound robustly to DI-DIII so did antibodies that recognize the DI-DIII linker region (MZ2, MZ4) (FIG. 3). MZ4-like antibodies do not bind to DI-DII and DIII fragments, nor to a DI-DIII linker synthetic peptide, as they only recognize the DI-DIII linker in its native context within the E protein. The fact that MZ4 binds strongly to the DI-DIII subunit described herein suggests that DI-DIII adopts a native-like conformation similar to the one it adopts when it is part of the full-length E protein, confirming that it is well folded in the absence of DII. As expected, the 2A10G6 antibody (Deng et al., PLOS One, 2011, 6 (1): e16059) that targets the FLE in DII did not bind DI-DIII.

iv. DI-DIII is Recognized by B Cells from Vaccinated Individuals

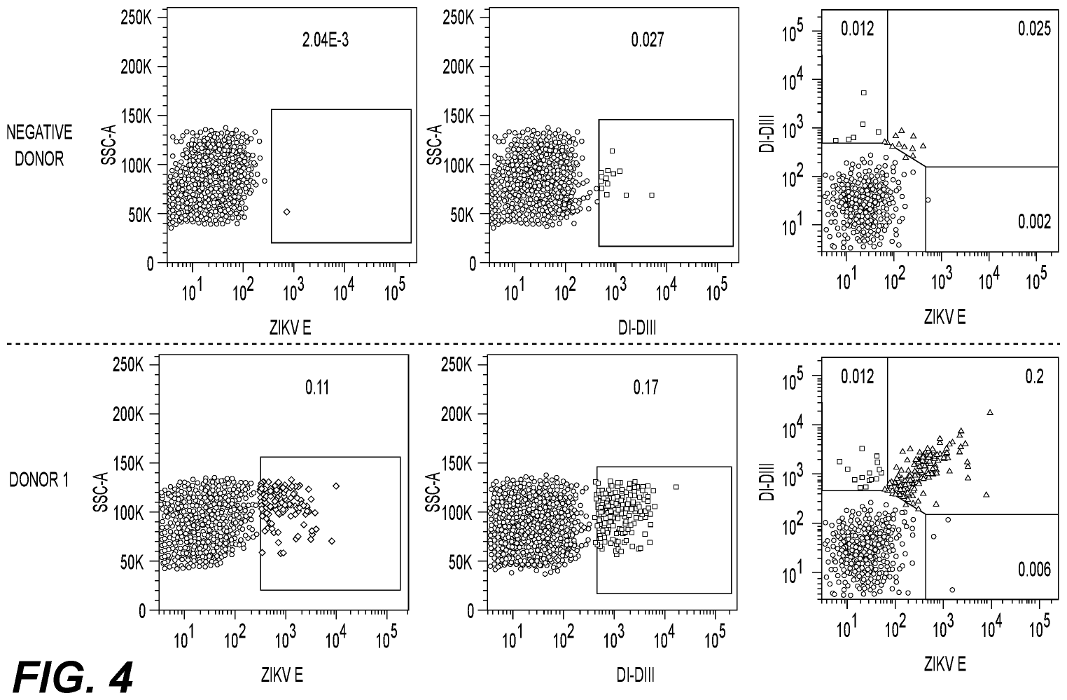

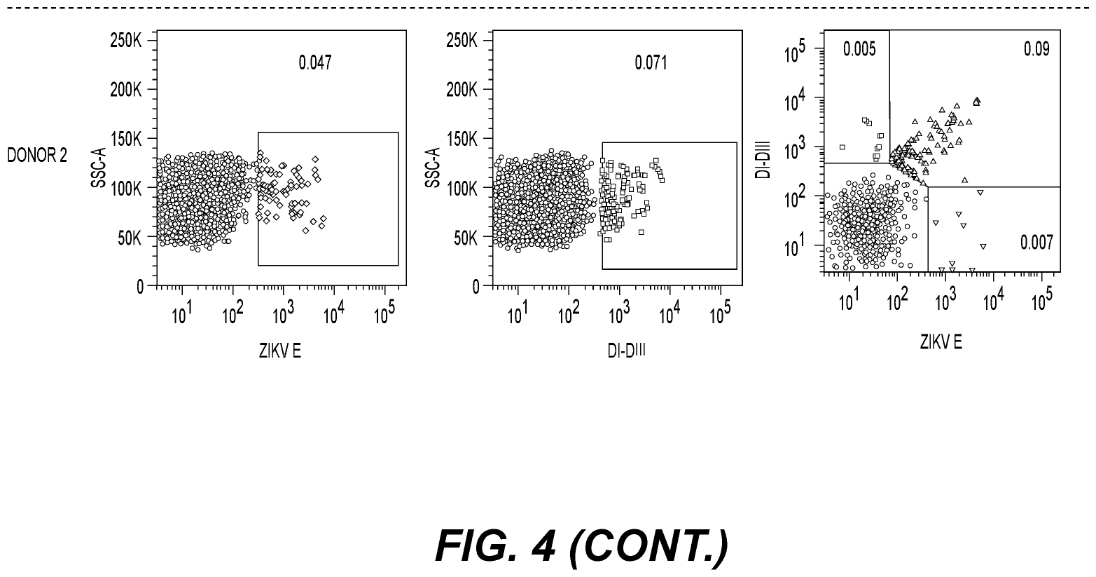

To further evaluate the antigenic properties of DI-DIII, its ability to react with memory B cells (via the membrane bound antibody also known as B cell receptor) from donors vaccinated with a whole inactivated ZIKV vaccine was assessed, where the breadth of the responses likely encompasses many more specificities than this study could have tested with monoclonal antibodies in the BLI assay used. Interestingly, in two different vaccinated donors, higher frequency of DI-DIII positive B cells compared to ZIKV E positive B cells was found (FIG. 4), indicating that DI-DIII would constitute a great probe or bait to interrogate B cell responses in humans and possibly isolate potent monoclonal antibodies from those B cells. Large populations of DI-DIII⁺/ZIK VE⁺ double positive B cells were also found, confirming that the engineered DI-DIII fragment is displaying many of the epitopes the full-length ZIKV E protein is presenting.

### Example 2. In Vivo Assessment on the Protective Effect of DI-DIII from Lethal ZIKV Infection

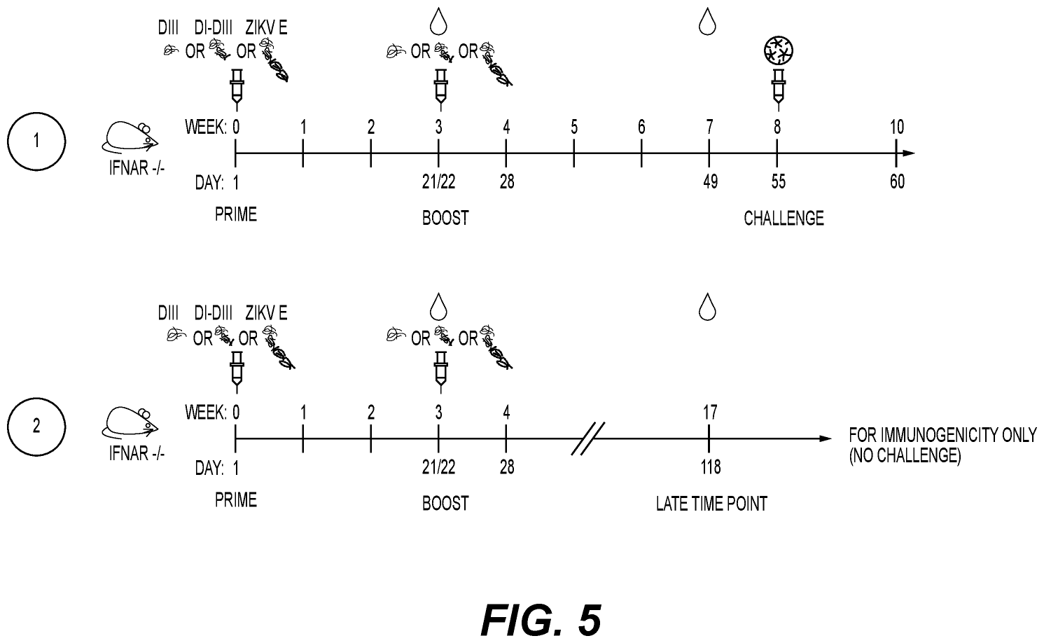

To assess whether DI-DIII could elicit protective responses again ZIKV infection in vivo, IFNAR^{-/-} mice were immunized with 5 μg of recombinant DI-DIII, DIII, or full-length ZIKV E protein, or vehicle and adjuvant-only controls, at day 1 and 21, followed by a subcutaneous challenge with 10⁵ FFU of ZIKV-PRVABC59 at day 55. A schematic of this prime/boost vaccination schedule is shown in FIG. 5 (top). A parallel experiment without ZIKV challenge was also conducted for immunogenicity study (FIG. 5, bottom).

Animals were monitored for weight loss and signs of clinical illness. This study was approved by the institutional Animal Care and Use Committee (IACUC), and research was conducted in compliance with the Animal Welfare Act and other federal statutes and regulations relating to animals.

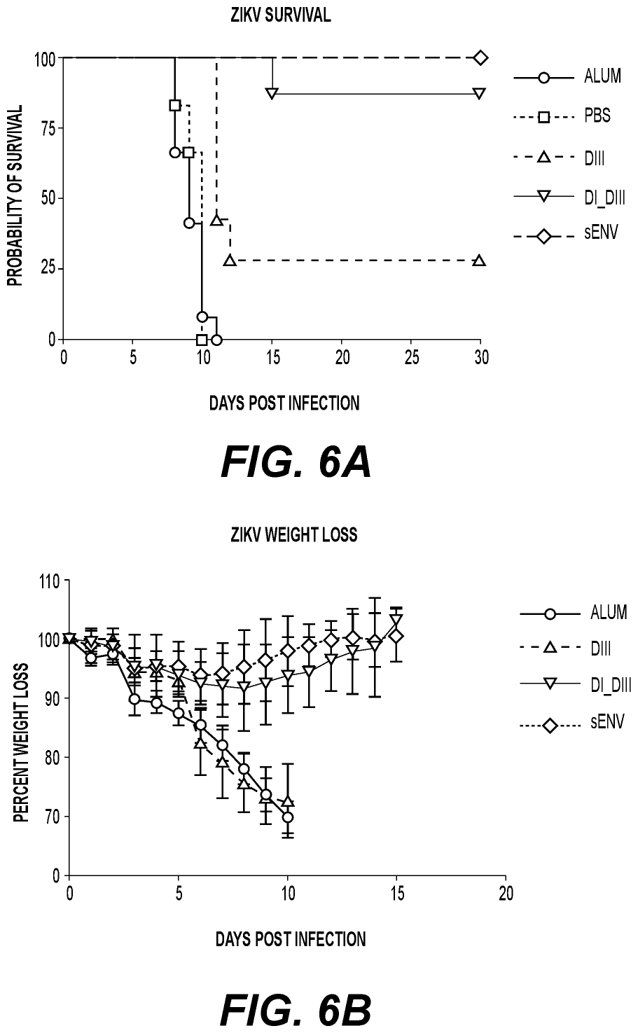

As shown in FIGS. 6A and 6B, two immunizations with DI-DIII protected mice from lethal ZIKV challenge, indicating that DI-DIII is immunogenic in mice and can elicit protective neutralizing antibody responses matching those of the full-length E protein.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1. An immunogen comprising Formula (I): 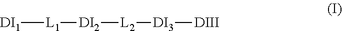
wherein DI₁, DI₂, and DI₃ together form Domain I (DI) of a flavivirus envelope (E) protein, DIII is Domain III (DIII) of the flavivirus E protein, and L₁ and L₂ are each independently a flexible linker, and wherein the immunogen does not comprise Domain II of the flavivirus E protein.

2. The immunogen of claim 1, wherein the flavivirus E protein is an E protein of Zika virus (ZIKV), dengue virus (DENV), Japanese Encephalitis Virus (JEV), West Nile Virus (WNV), Yellow Fever Virus (YFV), St Louis Encephalitis Virus (SLEV), Spondweni Virus (SPOV), Usutu Virus (USUV), Ilheus Virus (ILHV), Rocio Virus (ROCV), Wesselsbron Virus (WSLV), Tick Borne Encephalitis Virus (TBEV), Powassan Virus (POWV), Langat Virus (LGTV), Kyasanur Forest Disease (KSD), Alkhurma fever disease (AFD), or Omsk hemorrhagic fever virus (OHFV).

3. The immunogen of claim 2, wherein the flavivirus E protein comprises an amino acid sequence at least about 80% identical to the amino acid sequence of SEQ ID NO: 27.

4. The immunogen of claim 1, wherein;
a) DI₁ comprises an amino acid sequence at least about 90% identical to SEQ ID NO: 1, DI₂ comprises an amino acid sequence at least about 90% identical to SEQ ID NO: 2, DI₃ comprises an amino acid sequence at least about 90% identical to SEQ ID NO: 3, and DIII comprises an amino acid sequence at least about 90% identical to SEQ ID NO: 4; or
b) DI₁ comprises the amino acid sequence of SEQ ID NO: 1, DI₂ comprises the amino acid sequence of SEQ ID NO: 2, DI₃ comprises the amino acid sequence of SEQ ID NO: 3, and DIII comprises the amino acid sequence of SEQ ID NO: 4.

5. (canceled)

6. The immunogen of claim 1, wherein;
a) L₁ and L₂ each independently comprise one or more glycine residues and have a length of from about 4 to about 20 amino acids;
b) L₁ and L₂ are each independently selected from SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15;
c) L₁ and L₂ each comprises the amino acid sequence of SEQ ID NO: 5.

7-8. (canceled)

9. The immunogen of claim 1, further comprising one or more heterologous peptides linked to the C-terminus of the immunogen.

10. The immunogen of claim 9, wherein the one or more heterologous peptides comprise the amino acid sequence of SEQ ID NO: 16, SEQ ID NO: 17, and/or SEQ ID NO: 18.

11. The immunogen of claim 1, wherein:
a) the immunogen comprises an amino acid sequence at least about 95% identical to the amino acid sequence of SEQ ID NO: 19, SEQ ID NO: 20, or SEQ ID NO: 21; or
b) the immunogen comprises the amino acid sequence of SEQ ID NO: 19, SEQ ID NO: 20, or SEQ ID NO: 21.

12. (canceled)

13. A nucleic acid molecule encoding the immunogen of claim 1.

14. The nucleic acid molecule of claim 13, wherein the nucleic acid molecule is a DNA molecule or an RNA molecule.

15. The nucleic acid molecule of claim 14, wherein the RNA molecule is a messenger RNA (mRNA) molecule.

16. A composition comprising the immunogen of claim 1 or a nucleic acid molecule encoding said immunogen.

17. The composition of claim 16, wherein the composition is an immunogenic composition.

18. A vaccine comprising the composition of claim 17, and a pharmaceutically acceptable carrier.

19. The vaccine of claim 18, further comprising an adjuvant.

20. A method of immunizing a subject against a flavivirus infection, reducing one or more symptoms of a flavivirus infection in a subject, or inducing an immune response in a subject against flavivirus, the method comprising administering to the subject in need thereof the vaccine of claim 18 with or without an adjuvant.

21. The method of claim 20, wherein the method prevents a flavivirus infection in the subject, decreases the subject's likelihood of getting a flavivirus infection, reduces the subject's likelihood of getting serious illness from a flavivirus infection, or raises a protective immune response in the subject.

22. (canceled)

23. The method of claim 20, wherein:
a) the subject is a human;
b) the vaccine is administered intramuscularly, intradermally, subcutaneously, intravenously, intranasally, by inhalation, or intraperitoneally; and/or
c) the flavivirus is Zika virus (ZIKV), dengue virus (DENV), Japanese Encephalitis Virus (JEV), West Nile Virus (WNV), Yellow Fever Virus (YFV), St Louis Encephalitis Virus (SLEV), Spondweni Virus (SPOV), Usutu Virus (USUV), Ilheus Virus (ILHV), Rocio Virus (ROCV), Wesselsbron Virus (WSLV), Tick Borne Encephalitis Virus (TBEV), Powassan Virus (POWV), Langat Virus (LGTV), Kyasanur Forest Disease (KSD), Alkhurma fever disease (AFD), and/or Omsk hemorrhagic fever virus (OHFV).

24-27. (canceled)

28. A method of identifying an antibody against flavivirus in a sample, the method comprising:
a) contacting a sample with at least one polypeptide comprising an amino acid sequence at least about 95% identical to the amino acid sequence of SEQ ID NO: 19, SEQ ID NO: 20, or SEQ ID NO: 21; and
b) determining formation of a complex between the at least one polypeptide and at least one substance in the sample,
wherein formation of the complex indicates that the at least one substance is an antibody against flavivirus.

29. A method of identifying a B cell lymphocyte expressing an antibody that binds to an antigen of a flavivirus, the method comprising:
a) contacting a B cell lymphocyte in a sample with at least one polypeptide comprising an amino acid sequence at least about 95% identical to the amino acid sequence of SEQ ID NO: 19, SEQ ID NO: 20, or SEQ ID NO: 21; and
b) determining formation of a complex between the B cell lymphocyte and the at least one polypeptide,
wherein formation of the complex indicates that the B cell lymphocyte expresses an antibody that binds to an envelope (E) protein of the flavivirus,
wherein the at least one polypeptide is labeled with one or more chemicals that are able to emit fluorescence and the complex is determined using a fluorescence-activated cell sorting device, or
wherein the at least one polypeptide is labeled with one or more chemicals that are able to emit chemiluminescent light, and the complex is determined using a device that is capable of detecting chemiluminescent light.

30-32. (canceled)
